(19) United States
(12) Patent Application Publication
(10) Pub. No.: US 2019/0002420 A1
(43) Pub. Date: Jan. 3, 2019

(54) PROCESS AND INTERMEDIATES FOR PREPARATION OF THIAZINE DERIVATIVES

(71) Applicant: Shionogi & Co., Ltd., Osaka-shi, Osaka (JP)

(72) Inventors: Naohiro ONODERA, Toyonaka-shi, Osaka (JP); Kouichi NOGUCHI, Amagasaki-shi, Hyogo (JP); Shigeru ANDO, Toyonaka-shi, Osaka (JP); Daiki NAGAMATSU, Toyonaka-shi, Osaka (JP); Kenichi ISHIBASHI, Toyonaka-shi, Osaka (JP); Shunsuke OCHI, Amagasaki-shi, Hyogo (JP); Aiko HASEGAWA, Amagasaki-shi, Hyogo (JP); Katsuo ODA, Toyonaka-shi, Osaka (JP)

(73) Assignee: Shionogi & Co., Ltd., Osaka-shi, Osaka (JP)

(21) Appl. No.: 16/062,955

(22) PCT Filed: Dec. 22, 2016
(86) PCT No.: PCT/JP2016/088415

§ 371 Date: Jun. 15, 2018

(30) Foreign Application Priority Data
Dec. 25, 2015 (JP) 2015-254844

(51) Int. Cl. C07D 279/06; C07D 417/12

(52) U.S. Cl. CPC C07D 279/06; C07D 417/12

(57) ABSTRACT

The present invention provides a process for preparation of the compound of formula (VI), wherein each symbol is as defined in the specification, without using any intermediate compound showing mutagenicity. The process comprises salt formation of the intermediate compound of formula (I) with acid to enable optical resolution to isolate the intermediate compound of formula (II) in a stereo-selective manner.

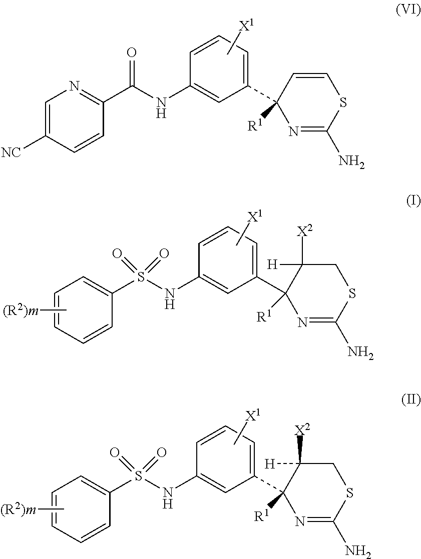

### FIELD

The present invention relates to a process for preparation of the compound of formula (VI):

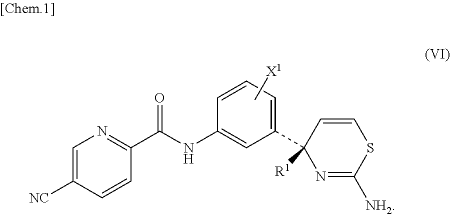

The present invention also relates to intermediates for preparation of the compound of formula (VI).

### BACKGROUND

The compound of formula (VI) has BACE1 inhibitory activity, and therefore, useful as a therapeutic agent for Alzheimer's disease (Patent Literature 1). The compound has a chiral center, which is assigned as S-configuration, at a carbon on the thiazine ring. Patent Literature 1 discloses a process for preparation of the compound of formula (VI) in a stereo-selective manner using a chiral intermediate compound.

Substituted-aminothiazine derivatives having a structure similar to that of formula (VI) were disclosed (Patent Literature 2). Also, substituted-aminooxazine derivatives were disclosed (Patent Literature 3). Patent Literature 3 exemplifies the formation of diastereoisomeric salts for obtaining the optical isomers as one of conventional methods. However, these compounds as disclosed were prepared by forming thiazine or oxazine ring through a cyclization of a chiral intermediate compound.

### CITATION LIST

### Patent Literature

### PTL 1: WO 2009/151098

### PTL 2: WO 2011/070781

### PTL 3: WO 2014/134341

### SUMMARY

### Technical Problem

The present invention provides a process for preparation of the compound of formula (VI) without using chiral intermediate compound as disclosed in prior art to form the thiazine ring. Also, the present invention provides intermediate compounds for preparation of the compound of formula (VI).

### Solution to Problem

The process provided by the present invention includes:

[1] A process for preparing a compound of formula (II) or a salt thereof,

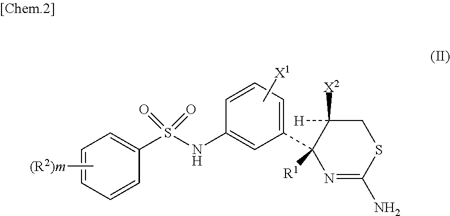

wherein
X¹ and X² are independently halogen;
R¹ is an optionally substituted alkyl;
R² is each independently NO₂, methyl, CF₃, halogen or methyloxy; and
m is an integer of 1 or 2

which comprises subjecting a compound of formula (I) or a salt thereof,

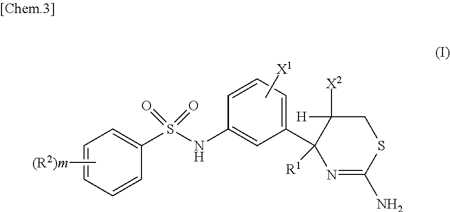

wherein each symbol is as defined above, to optical resolution using (L)-tartaric acid or (D)-malic acid;

[2] The process of [1] wherein the optical resolution is carried out in a mixed solvent comprising water and one or more organic solvent selected from the group consisting of acetonitrile, methanol, 2-propanol, butanol, ethyl acetate, ethyl formate, acetone and methyl ethyl ketone;

[3] The process of [2] wherein the mixed solvent comprises water, 2-propanol and ethyl acetate;

[4] A process for preparing crystals of an acetate salt of a compound of formula (IV),

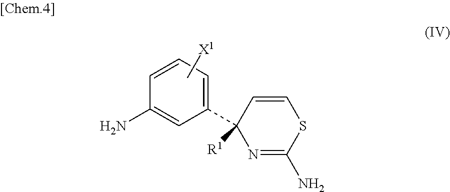

wherein
X¹ is halogen; and
R¹ is an optionally substituted alkyl,

which comprises the steps of:
treating a compound of formula (III) or a salt thereof,

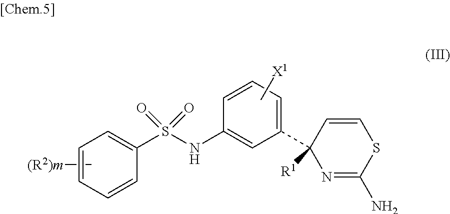

wherein each symbol is as defined above under acidic condition; and
subjecting the product to crystallization with acetic acid;

[5] The process of [4] wherein the compound of formula (III) is that obtained by subjecting a compound of formula (II) or a salt thereof obtained by the process of any one of [1] to [3] to dehydrohalogenation reaction;

[6] A process for preparing a compound of formula (VI) or a salt thereof,

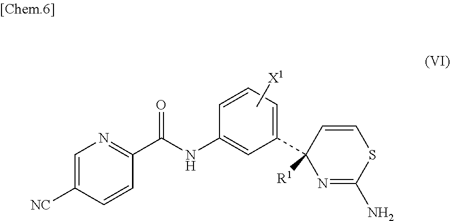

wherein
X¹ is halogen; and
R¹ is an optionally substituted alkyl,

which comprises reacting the acetate salt of a compound of formula (IV) obtained by the process of [4] or [5] or a free form thereof,

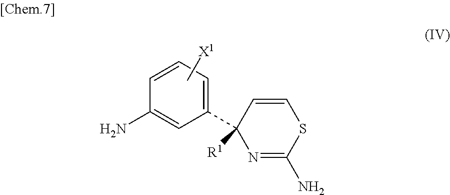

wherein each symbol is as defined above,

with a compound of formula (g):

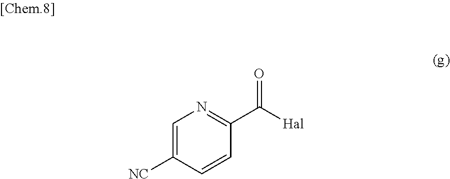

wherein Hal is halogen;

[7] A process for preparing a compound of formula (VI) or a salt thereof,

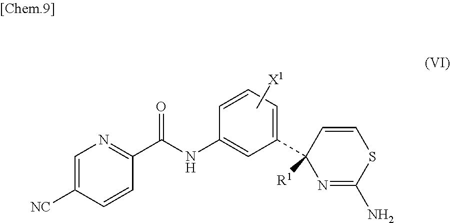

wherein
X¹ is halogen; and
R¹ is an optionally substituted alkyl,

which comprises the steps:

(1) subjecting a compound of formula (II) or a salt thereof to dehydrohalogenation reaction,

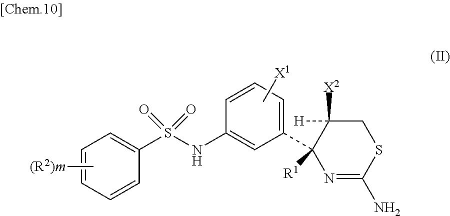

wherein
X¹ and R¹ are as defined above;
X² is halogen;
R² is each independently NO₂, methyl, CF₃, halogen or methyloxy; and
m is an integer of 1 or 2;

(2) deprotecting the product of formula (III) or a salt thereof,

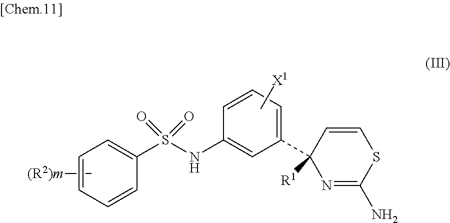

wherein each symbol is as defined above,

and

(3) reacting the product of formula (IV) or a salt thereof:

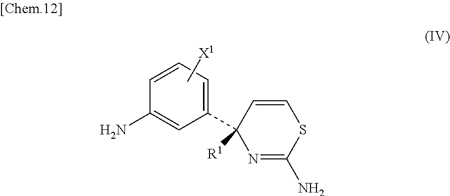

wherein each symbol is as defined above,

with a compound of formula (g):

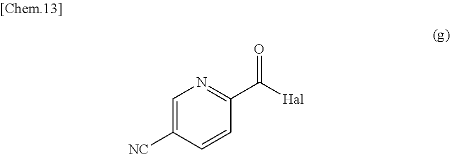

wherein Hal is as defined above;

[8] The process of [7] wherein the compound of formula (II) or a salt thereof is obtained by the process of any one of [1] to [3].

[9] A process for preparing a crystal of compound of formula (VI) or a salt thereof,

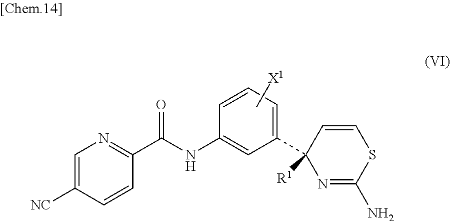

wherein
X¹ is halogen; and
R¹ is an optionally substituted alkyl,

which comprises the compound (VI) obtained by the process of any one of [6] to [8] is neutralized with organic base having 8 or more of pKa;

[10] The process of [9] wherein the base is selected from the group consisting of alkylamine, dicyclohexylamine, ethanolamine, diethanolamine, triethanolamine, meglumine, ethylenediamine, and the mixture thereof.

[11] The process of [9] or [10] wherein the base is triethylamine.

[12] A compound of formula (VII) or a salt thereof,

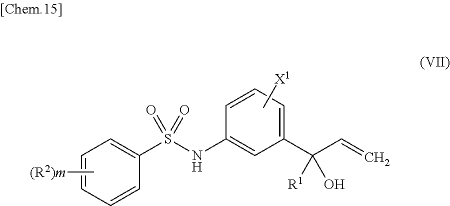

wherein
X¹ is halogen;
R¹ is an optionally substituted alkyl;
R² is each independently NO₂, methyl, CF₃, halogen or methyloxy; and
m is an integer of 1 or 2;

[13] A compound of formula (VIII) or a salt thereof,

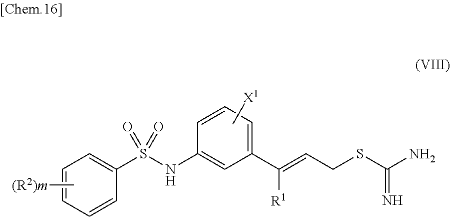

wherein
X¹ is halogen;
R¹ is an optionally substituted alkyl;
R² is each independently NO₂, methyl, CF₃, halogen or methyloxy; and
m is an integer of 1 or 2;

[14] A compound of formula (II) or a salt thereof,

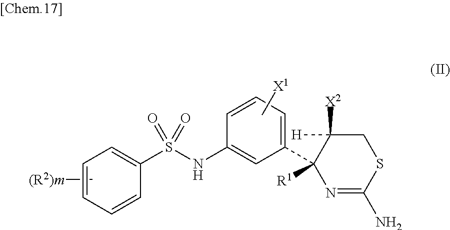

wherein
X¹ and X² are independently halogen;
R¹ is an optionally substituted alkyl;
R² is each independently NO₂, methyl, CF₃, halogen or methyloxy; and
m is an integer of 1 or 2;

[15] The salt of [14] which is tartrate salt or malate salt;

[16] A salt of a compound of formula (IV):

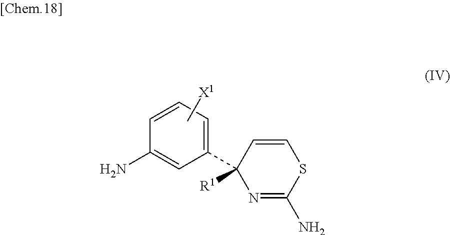

X¹ is halogen; and

R¹ is an optionally substituted alkyl; and

[17] The salt of [16] which is acetate salt.

### BRIEF DESCRIPTION OF DRAWINGS

FIG. 1 represents the powder X-ray diffraction pattern of the crystal of Compound 9. The x-axis shows the 2-theta value and the y-axis the intensity (Count).

FIG. 2 represents the results of thermogravimetry/differential thermal analysis (TG/DTA) analysis of the crystal of Compound 9.

FIG. 3 represents the results of dynamic vapor sorption (DVS) analysis of the crystal of Compound 9.

FIG. 4 represents the powder X-ray diffraction pattern of the crystal of Compound 11.

FIG. 5 represents the powder X-ray diffraction pattern of the crystal Form I of Compound 15.

FIG. 6 represents the powder X-ray diffraction pattern of the crystal Form II of Compound 15.

FIG. 7 represents the drawing of Molecule I using PLATON/ORTEP.

FIG. 8 represents the drawing of Molecule II using PLATON/ORTEP.

### DESCRIPTION OF EMBODIMENTS

The respective terms used herein are as defined alone or in combination with other terms as follows.

The term “halogen” includes fluorine, chlorine, bromine and iodine. Preferably, “halogen” for X¹ is fluorine. Preferably, “halogen” for X² is bromine.

The term “alkyl” includes straight or branched alkyls of a carbon number of 1 to 8, preferably 1 to 6, and further preferably 1 to 3. Examples of “alkyl” include methyl, ethyl, n-propyl, isopropyl, n-butyl, isobutyl, sec-butyl, tert-butyl, n-pentyl, isopentyl, neopentyl, hexyl, isohexyl, n-heptyl, isoheptyl, and n-octyl.

Examples of the substituent of “optionally substituted alkyl” include same or different one or more group(s), preferably 1 to 3 group(s) selected from halogen such as fluorine.

Examples of “optionally substituted alkyl” include, but are not limited to, methyl, fluoromethyl, difluoromethyl and trifluoromethyl.

Examples of

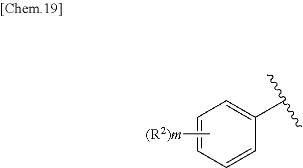

include

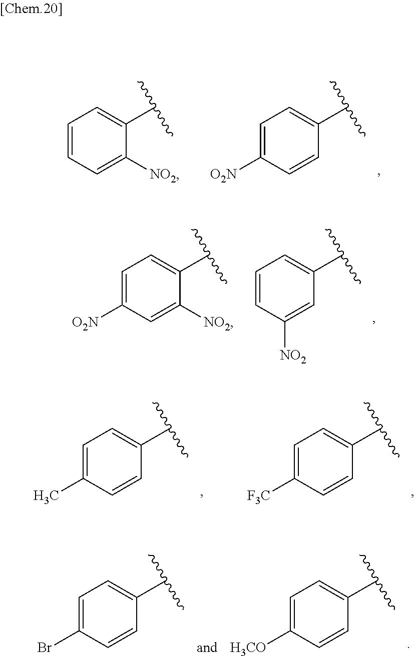

Preferable examples include

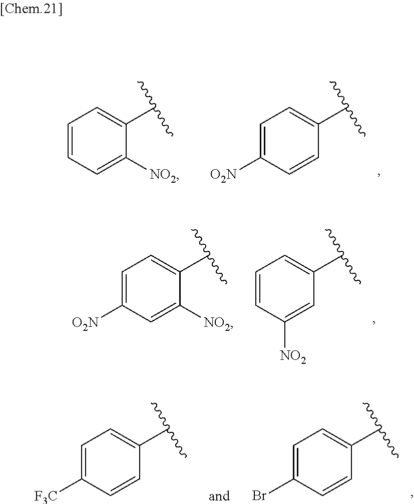

and more preferable examples include

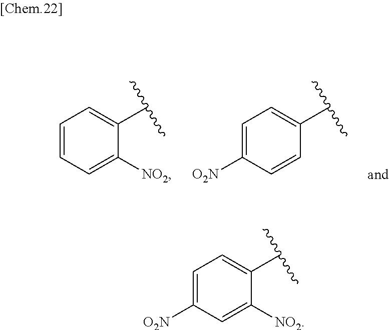 [SMILES: *c1ccccc1[N+](=O)[O-] ; *c1ccc([N+](=O)[O-])cc1 ; "and" ; *c1ccc([N+](=O)[O-])cc1[N+](=O)[O-]]

One or more hydrogen, carbon and/or other atoms in the compounds according to the present invention may be replaced with isotopes of hydrogen, carbon and/or other atoms respectively. Examples of isotopes include, but are not limited to, isotopes of hydrogen, carbon, nitrogen, oxygen, phosphorus, sulfur, fluorine, iodine and chlorine, such as ²H, ³H, ¹¹C, ¹³C, ¹⁴C, ¹⁵N, ¹⁸O, ¹⁷O, ³¹P, ³²P, ³⁵S, ¹⁸F, ¹²³I and ³⁶Cl. The compounds according to the present invention include compounds replaced with these isotopes. The compounds replaced with the above isotopes are useful as medicines and include all of radiolabeled compounds of the compound herein described. A “method of radiolabeling” in the manufacture of the “radiolabeled compounds” is encompassed by the present invention, and the resultant “radiolabeled compounds” are useful in studies on metabolized drug pharmacokinetics, studies on binding assay and/or as a diagnostic tool.

A radiolabeled compound herein described can be prepared using well-known methods in this field of the invention. For example, a tritium-labeled compound herein described can be prepared by introducing a tritium into a certain compound herein described, through a catalytic dehalogenation reaction using a tritium. This method comprises reacting with an appropriately-halogenated precursor of the compound herein described with tritium gas in the presence of an appropriate catalyst, such as Pd/C, and in the presence or absent of a base. The other appropriate method of preparing a tritium-labeled compound can be referred to “Isotopes in the Physical and Biomedical Sciences, Vol. 1, Labeled Compounds (Part A), Chapter 6 (1987)”, the entire contents of which are hereby incorporated by reference. A ¹⁴C-labeled compound can be prepared by using a raw material having ¹⁴C.

The salts of the compounds according to the present invention include, for example, salts with alkaline metal (e.g., lithium, sodium, potassium or the like), alkaline earth metal (e.g., calcium, barium or the like), magnesium, transition metal (e.g., zinc, iron or the like), ammonia, organic bases (e.g., trimethylamine, triethylamine, dicyclohexylamine, ethanolamine, diethanolamine, triethanolamine, meglumine, ethylenediamine, pyridine, picoline, quinoline or the like) or amino acids, or salts with inorganic acids (e.g., hydrochloric acid, sulfuric acid, nitric acid, carbonic acid, hydrobromic acid, phosphoric acid, hydroiodic acid or the like) or organic acids (e.g., formic acid, acetic acid, propionic acid, trifluoroacetic acid, citric acid, lactic acid, tartaric acid, oxalic acid, maleic acid, fumaric acid, mandelic acid, glutaric acid, malic acid, benzoic acid, phthalic acid, ascorbic acid, benzenesulfonic acid, p-toluenesulfonic acid, methanesulfonic acid, ethanesulfonic acid or the like). Especially, salts with hydrochloric acid, sulfuric acid, phosphoric acid, tartaric acid, malic acid, methanesulfonic acid and the like are included. These salts can be formed by methods well known in the art.

The compounds according to the present invention or salts thereof may form solvates, such as hydrates or the like, cocrystal and/or crystal polymorphs. The compounds according to the present invention encompasses those various solvates, cocrystal and crystal polymorphs. “Solvates” may be those wherein any numbers of solvent molecules, such as water molecules or the like, are coordinated with the compounds. When the compounds or salts thereof are allowed to stand in the atmosphere, the compounds may absorb water, resulting in attachment of adsorbed water or formation of hydrates. Recrystallization of the compounds or salts thereof may produce crystal polymorphs. The term “cocrystal” means that a compound or salt thereof and a counter-molecule exist in the same crystal lattice, and it can be formed with any number of counter-molecules.

The following Scheme 1 describes an exemplary process of the invention to prepare Compound (VI).

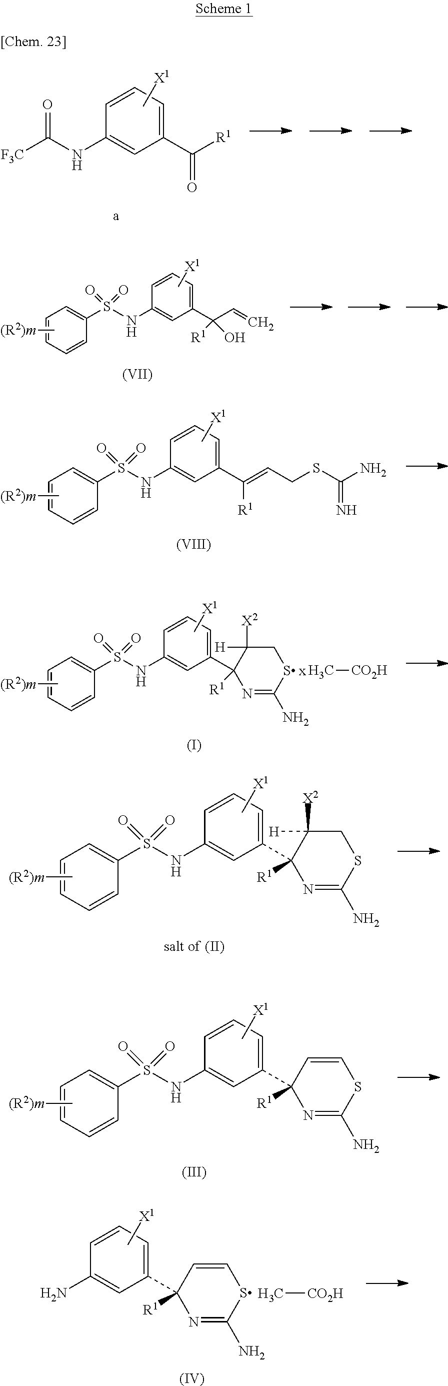

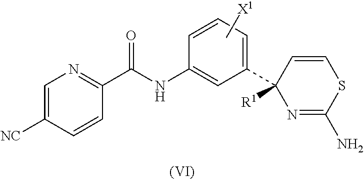

wherein X¹, X², R¹, R² and m are as defined above.

The process of the present invention features introducing an arylsulfonyl group, such as a nosyl group, as an amino protecting group in the intermediates Compounds (VII), (VIII), (I), (II) and (III) as shown above. Also, the process features salt formation with acid such as (L)-tartaric acid or (D)-malic acid to give a salt of the intermediate Compound (II) to enable optical resolution of the intermediate compound.

Additionally, the process features preparing crystals of an acetate salt of a compound of formula (IV).

In one embodiment of the invention, the intermediate Compound (VIII) is prepared as shown in Scheme 1-A.

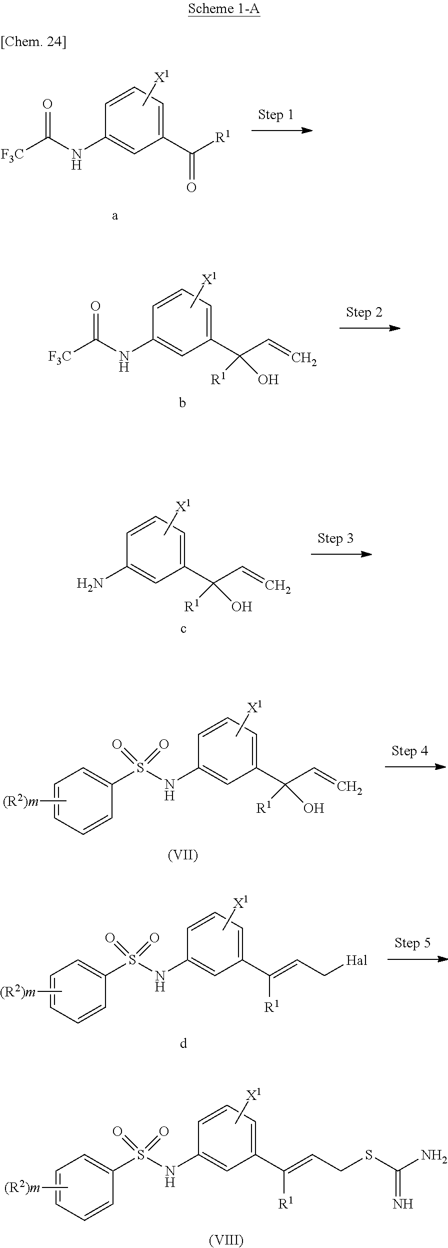

wherein Hal is halogen and X¹, R¹, R² and m are as defined above.

The starting Compound (a) is commercially available or may be prepared from commercially available material by methods well known in the art.

(Step 1) Vinylation of Compound (a) gives Compound (b). The step is carried out using a Grignard reagent such as vinylmagnesium chloride (VMC) according to known method such as those described in WO 2008/133274.

(Step 2) Compound (b) is hydrolyzed to afford Compound (c). The step is carried out using a strong base, such as NaOH, KOH, Na₂CO₃, K₂CO₃, Cs₂CO₃ and LiOH, under suitable conditions in a suitable solvent, such as methanol, ethanol, 1-butanol, toluene, dioxane, acetonitrile, diethyl ether, THF, DMSO, DMF, N-methylpyrrolidone, N,N-dimethylacetamide, dichloromethane, water, and a mixture thereof, to give Compound (c).

(Step 3) Compound (c) is protected to give Compound (VII) in which the amino group is protected by an arylsulfonyl group such as a nosyl group. The step is carried out using arylsulfonyl halide such as 2-nitrobenzenesulfonyl chloride, 2-nitrobenzenesulfonyl bromide, 2-nitrobenzenesulfonyl iodide, 4-nitrobenzenesulfonyl chloride, 2,4-dinitrobenzenesulfonyl chloride, 3-nitrobenzenesulfonylchloride, 4-methoxybenzeneslfonyl chloride, 4-(trifluoromethyl)benzenesulfonyl chloride, 4-bromobenzenesulfonyl chloride, and a base, such as sodium hydrogen carbonate, sodium carbonate, potassium carbonate, NaOH, KOH, triethylamine, trimethylamine, pyridine, N-methylmorpholine under suitable conditions in a suitable solvent, such as toluene, ethyl acetate, dioxane, acetonitrile, diethyl ether, THF, DMSO, DMF, N-methylpyrrolidone, N,N-dimethylacetamide, dichloromethane, water and a mixture thereof. The reaction temperature is preferably 0° C. to 100° C., preferably 30° C. to 70° C., and more preferably around 50° C. In case where Compound (c) is protected with trifluoroacetyl group (CF₃ CO—), which is commonly used as an amino protecting group, the obtained protected compound (CF₃CO protected derivative) is unstable and degradable, while Compound (VII) of the invention is stable. Thus, Compound (VII) is useful in the process of the invention for the production of the pharmaceutical compound of formula (VI).

(Step 4) Compound (VII) is halogenated to afford Compound (d). The step is carried out according to known methods in the art, such as those described in WO2008/133274, using hydrochloric acid under suitable conditions in a suitable solvent, such as methanol, ethanol, 1-butanol, toluene, ethyl acetate, dioxane, acetonitrile, diethyl ether, THF, DMSO, DMF, N-methylpyrrolidone, N,N-dimethylacetamide, dichloromethane, water, and a mixture thereof. The reaction temperature is preferably 0° C. to 100° C., preferably 0° C. to 40° C., and more preferably around room temperature.

(Step 5) Compound (d) is reacted with thiourea to afford Compound (VIII). The step is carried out according to known methods in the art, such as those described in WO2008/133274, under suitable conditions in a suitable solvent, such as methanol, ethanol, 1-butanol, toluene, ethyl acetate, dioxane, acetonitrile, diethyl ether, THF, DMSO, DMF, N-methylpyrrolidone, N,N-dimethylacetamide, dichloromethane, water, and a mixture thereof. The reaction temperature is preferably 0° C. to 100° C., preferably 30° C. to 70° C., and more preferably around 50° C.

In case hydrochloric acid is used in Step 4, the formation of hydrochloride salt may occur in the step, and the precipitated salt may be isolated. The hydrochloride salt thus obtained is treated with a base, such as NaOH, KOH, Na₂CO₃, K₂CO₃, Cs₂CO₃ and LiOH, to obtain Compound (VIII) under suitable conditions in a suitable solvent, such as methanol, ethanol, 1-butanol, toluene, ethyl acetate, dioxane, acetonitrile, diethyl ether, THF, DMSO, DMF, N-methylpyrrolidone, N,N-dimethylacetamide, dichloromethane, water and a mixture thereof. The compound may be crystallized to isolate. The crystallization of the compound is well known and appreciated in the art.

In one embodiment of the invention, the intermediate Compound (II) is prepared as shown in Scheme 1-B.

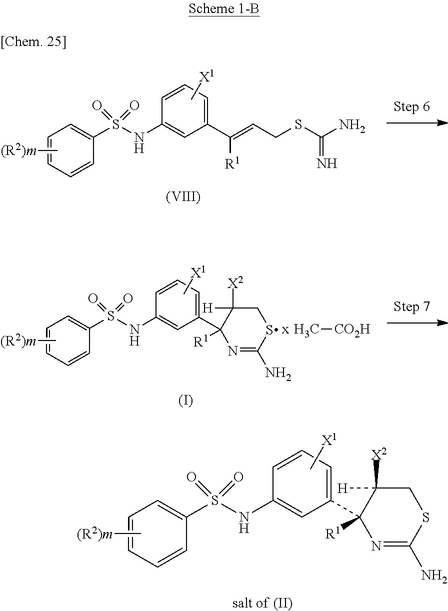

wherein X¹, X², R¹, R² and m are as defined above.

(Step 6) Compound (VIII) obtained above is cyclized to afford Compound (I). The step is carried out using an acid such as acetic acid and N-halosuccinimide under suitable conditions in a suitable solvent, such as toluene, ethyl acetate, dioxane, acetonitrile, diethyl ether, THF, DMSO, DMF, N-methylpyrrolidone, N,N-dimethylacetamide, dichloromethane and a mixture thereof.

N-halosuccinimide is preferably N-bromosuccinimide. The reaction temperature is preferably −70° C. to room temperature, preferably −40° C. to 0° C., and more preferably around −20° C. The formation of acetate salt occurs in the step, and the precipitated salt may be isolated.

(Step 7) Compound (I) is treated with suitable acid to form a salt of a compound of formula (II). The salt of the compound of formula (II) (4R,5R-configuration) can be obtained stereo-selectively by optical resolution. The salt of the compound of formula (II) may be obtained as a solvate thereof, such as hydrate.

In a specific embodiment of the invention, suitable acid such as (L)-tartaric acid or (D)-malic acid is added to a solution of Compound (I), which is racemate, to form crystalline diastereomeric salt (4R,5R-configuration) of compound (I), which is then separated by a fractional crystallization to obtain the salt such as tartrate salt or malate salt of the compound of formula (II).

In one embodiment of the invention, the diastereomeric salt of the compound of formula (II) is crystallized in a solvent such as acetonitrile, methanol, ethanol, 2-propanol, butanol such as 1-butanol and 2-butanol, methyl acetate, ethyl acetate, ethyl formate, acetone, methyl ethyl ketone, methyl isobutyl ketone, methoxymethane, 2-Ethoxyethanol, dimethylacetoamide and water.

In another embodiment of the invention, the diastereomeric salt of the compound of formula (II) is crystallized in a mixed solvent comprising water and at least one organic solvent. Examples of organic solvent include, but are not limited to, one or more organic solvents selected from the group consisting of acetonitrile, methanol, ethanol, 2-propanol, butanol such as 1-butanol and 2-butanol, methyl acetate, ethyl acetate, ethyl formate, acetone and methyl ethyl ketone, methyl isobutyl ketone, methoxymethane, 2-ethoxyethanol and dimethylacetoamide.

More specifically, examples of organic solvent include one or more organic solvents selected from the group consisting of

acetonitrile, methanol, 2-propanol, butanol, ethyl acetate, ethyl formate, acetone, and methyl ethyl ketone.

In one preferred embodiment of the invention, the mixed solvent comprises water and one organic solvent selected from the group consisting of acetonitrile, methanol, 2-propanol, butanol, ethyl acetate, ethyl formate, acetone and methyl ethyl ketone.

In one embodiment of the invention, the ratio of water:acetonitrile is around 10:90 to 25:75, 50:50, or 75:25.

In one embodiment of the invention, the ratio of water:methanol is 80:20.

In one embodiment of the invention, the ratio of water:2-propanol is around 10:90 to 40:60.

In one embodiment of the invention, the ratio of water:butanol is around 25:75 to 75:25.

In one embodiment of the invention, the ratio of water:ethyl acetate is around 20:80.

In one embodiment of the invention, the ratio of water:ethyl formate is around 10:90 to 75:25.

In one embodiment of the invention, the ratio of water:acetone is around 80:20.

In one embodiment of the invention, the ratio of water:methyl ethyl ketone is around 10:90 to 25:75, 50:50 or 75:25.

In yet another preferred embodiment of the invention, the mixed solvent comprises water, 2-propanol and ethyl acetate.

In another embodiment of the invention, the mixed solvent comprises water, 2-propanol and ethyl acetate, and the ratio of water:2-propanol:ethyl acetate is around 20 to 40:30 to 50:20 to 50 (v/v), for example, around 20:40:40 (v/v) or 20:30:50 (v/v).

Example of the ratio of water:ethyl acetate is around 1:1.5 to 2.5 (v/v), for example, around 1:1.5 (v/v), 1:2 (v/v) or 1:2.5 (v/v).

Example of the ratio of water:2-propanol is around 1:1.5 to 2.5 (v/v), for example, around 1:1.5 (v/v), 1:2 (v/v) or 1:2.5 (v/v).

The ratio of Compound (II) having 4R,5R-configuration can be determined by analytical techniques known in the art such as HPLC. Also, the crystal form and structure of the obtained crystals can be determined by analytical techniques known in the art such as powder X-ray diffraction analysis, dynamic vapor sorption (DVS) analysis, and differential scanning calorimetry (DSC), etc.

In one embodiment of the invention, the intermediate Compound (IV) is prepared as shown in Scheme 1-C.

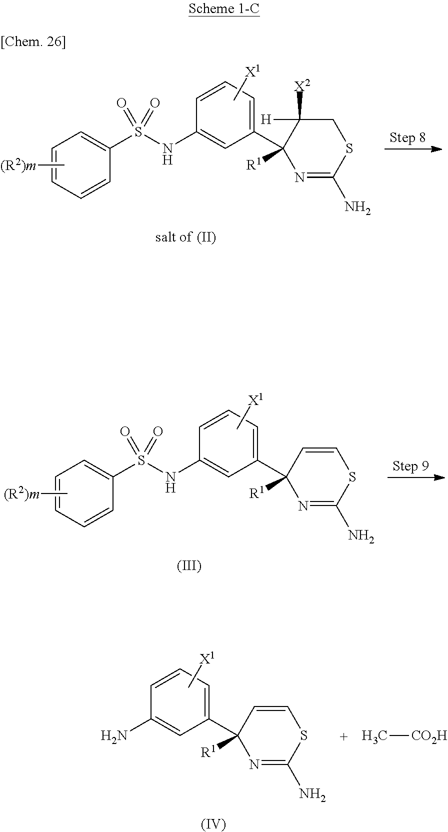

wherein X¹, X², R¹, R² and m are as defined above.

(Step 8) The salt of a compound of formula (II) or a solvate thereof such as hydrate is subjected to dehydrohalogenation reaction to afford Compound (III). The step is carried in the presence of a base, such as diazabicycloundecene, diazabicyclononene, triethylamine, trimethylamine, dimethylaniline, N-methylmorpholine, sodium t-butoxide, potassium t-butoxide, sodium t-pentoxide and potassium t-pentoxide, under suitable conditions in a suitable solvent, such as toluene, ethyl acetate, dioxane, acetonitrile, diethyl ether, THF, DMSO, DMF, N-methylpyrrolidone, N,N-dimethylacetamide, dichloromethane, and a mixture thereof at a temperature of −50° C. to 50° C., preferably −20° C. to room temperature, and more preferably around 5° C.

(Step 9) The protective group of Compound (III) is removed with a deprotecting agent such as thiol and a base such as sodium hydrogen carbonate, sodium carbonate, potassium carbonate, triethylamine, trimethylamine, N-methylmorpholine, and pyridine in an appropriate solvent such as methanol, ethanol, 1-butanol, toluene, ethyl acetate, dioxane, acetonitrile, diethyl ether, THF, DMSO, DMF, N-methylpyrrolidone, N,N-dimethylacetamide, dichloromethane, water, and a mixture thereof. The reaction is preferably carried at a temperature of 0° C. to 100° C., preferably 20° C. to 60° C., and more preferably around 40° C. Examples of the deprotecting agent include, but are not limited to, 4-chlorobenzenethiol, methanethiol, ethanethiol, propanethiol, dodecanethiol, and benzenethiol. The obtained product Compound (IV) may be used directly for the next step or crystalized with appropriate acid, such as but not limited to acetic acid, to afford a crystalline salt thereof as shown above. The crystallization is preferably carried out at a temperature of 0° C. to 20° C., and more preferably around 5° C. The acetate salt (IV) is effectively crystalized by adding acetic acid in an appropriate solvent to crystallize the salt. Examples of the solvent for use in the crystallization of the salt of a compound of formula (IV) include, but are not limited to, water, and organic solvents such as acetonitrile, methanol, 2-propanol, butanol, ethyl acetate, ethyl formate, acetone and methyl ethyl ketone. Acetonitrile, 2-propanol and ethyl acetate are especially preferred.

The crystalline structure of the obtained crystals can be determined by using any one of analytical techniques known in the art such as powder X-ray diffraction analysis, dynamic vapor sorption (DVS) analysis, and differential scanning calorimetry (DSC), etc.

In one embodiment of the invention, the intermediate Compound (VI) is prepared as shown in Scheme 1-D.

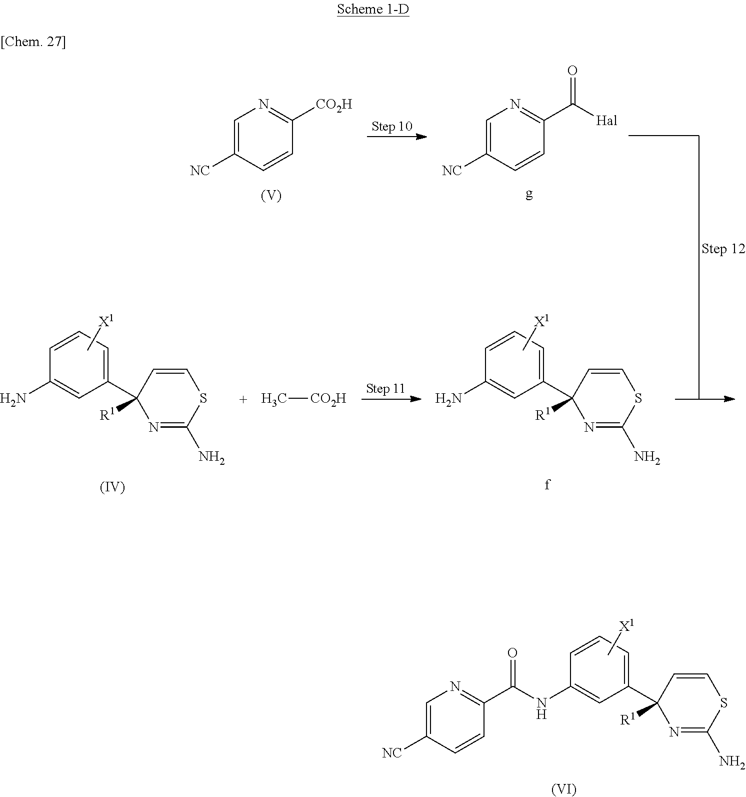

wherein Hal is halogen and X¹ and R¹ are as defined above.

(Step 10) Compound (V) is commercially available or may be prepared from commercially available material by methods well known in the art.

Compound (V) is halogenated using a halogenating agent in an appropriate solvent to afford Compound (g). Examples of the solvent include, but are not limited to, N-methylpyrrolidone, DMF, DMSO, THF, toluene, N,N-dimethylacetamide, dichloromethane and a mixture thereof. Preferred examples of the halogenating agent include thionyl chloride and oxalyl chloride. The step is preferably carried at a temperature of 0° C. to room temperature, and preferably around 5° C.

(Step 11) The salt of a compound of formula (IV) obtained above is treated with a base in a suitable solvent to afford Compound (f), which is a free form of Compound (IV). Examples of the solvent include, but are not limited to, methanol, ethanol, 1-butanol, toluene, ethyl acetate, dioxane, acetonitrile, diethyl ether, THF, DMSO, DMF, N-methylpyrrolidone, N,N-dimethylacetamide, dichloromethane, water, and a mixture thereof. Examples of the base include sodium hydrogen carbonate, sodium carbonate, potassium carbonate, NaOH, and KOH. The step is preferably carried at a temperature of 0° C. to 40° C., and more preferably around room temperature.

(Step 12) Compound (f) is reacted with Compound (g) to afford Compound (VI). Crystal of Compound (VI) may be obtained after neutralization with an appropriate base. Examples of such base include, but not limited to, sodium hydroxide, sodium hydrogen carbonate, sodium carbonate, potassium carbonate, triethylamine, trimethylamine, diisopropylethylamine, tributylamine, diisopropylamine, dicyclohexylamine, ethanolamine, diethanolamine, triethanolamine, meglumine, ethylenediamine, N-methylmorpholine, and pyridine, and a mixture thereof. An organic base having 8 or more of pKa is preferred to obtain a stable crystalline form of compound (VI) efficiently. Examples of such base include, but not limited to, alkylamine such as monoalkylamine, dialkylamine or trialkylamine, dicyclohexylamine, ethanolamine, diethanolamine, triethanolamine, meglumine, ethylenediamine, and the mixture thereof. Preferable examples include alkylamine such as triethylamine, trimethylamine, diisopropylethylamine, tributylamine, diisopropylamine, and the mixture thereof. More preferable is triethylamine.

The step is preferably carried in a suitable solvent, such as N-methylpyrrolidone, N,N-dimethylacetamide, DMF, DMSO, THF, acetonitrile, toluene, dichloromethane, ethyl acetate, water, and a mixture thereof, at a temperature of −20° C. to room temperature, and preferably around 3° C.

In another embodiment of the invention, Compound (VI) may be obtained by subjecting the salt of a compound of formula (IV) directly to the reaction with Compound (g) in a same manner as described in Step 12.

If necessary, Compound (VI) thus obtained may be recrystallized and purified according to known methods in the art.

The crystalline structure of the crystals of the compounds as obtained above can be determined by using any one of analytical techniques known in the art such as powder X-ray diffraction analysis, dynamic vapor sorption (DVS) analysis, and differential scanning calorimetry, etc. The method and conditions for caring out the analysis are well known and appreciated in the art.

The present invention shall be explained in more detail in the following examples with reference to the figures, but without being limited thereto.

The following abbreviations as used herein represent the following and should assist in understanding the invention:

THF—tetrahydrofuran

DMF—N,N-dimethylformamide

DMSO—Dimethyl sulfoxide

MeCN—acetonitrile

EtOAc—ethyl acetate

MeOH—methanol

PTEE—polytetrafluoroethylene

HPLC—high performance liquid chromatography

### EXAMPLES

### Example 1-1

Preparation of Compound 7

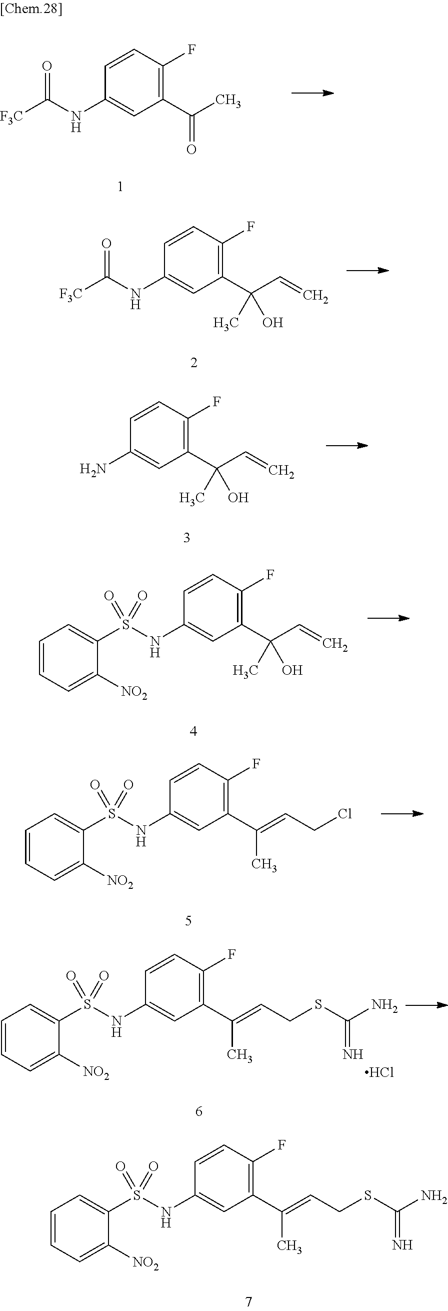

Compound 1 (30.0 g, 120.5 mmol) in tetrahydrofuran (165 mL) was added to 1.5 M vinylmagnesium chloride in tetrahydrofuran (297 mL, 445.9 mmol). The mixture was stirred for 1 hour at −20° C. and added to a mixture of toluene (120 mL), acetic acid (28.9 g, 481.5 mmol) and water (150 mL) to separate layers. To the organic layer was added 12% aqueous sodium hydroxide (120.1 g, 362.4 mmol), and the solution was stirred for 2.5 hours at 45° C. The layers were separated, and the organic layer was concentrated to 119 g. To the concentrated liquid were added tetrahydrofuran (30 mL), sodium bicarbonate (15.2 g, 181.2 mmol) and water (90 mL), and the mixture was warmed to 50° C. 25% nosyl chloride in toluene (117.4 g, 132.5 mmol) was added, and the mixture was stirred for 2.5 hours. The layers were separated, and the organic layer was concentrated to 93.4 g to obtain a concentrated solution of Compound 4.

To the concentrated solution of Compound 4 (84.0 g) were added ethyl acetate (81 mL) and 35% hydrochloric acid (45.2 g, 433.6 mmol), and the mixture was stirred for 2.5 hours at 25° C. Toluene (27 mL) and water (27 mL) were added to separate layers. The organic layer was added to a suspension of thiourea (10.7 g) in 1-butanol (16.1 g), and the mixture was stirred for 2.8 hours at 50° C. The mixture was cooled to 25° C., stirred for 1 hour and then filtered to obtain 42.6 g of crystals (wet crystals) of Compound 6.

To the wet crystals of Compound 6 (41.0 g) were added toluene (39 mL), ethyl acetate (117 mL), 1-butanol (52 mL) and water (130 mL). The mixture was cooled to 5° C. 6% aqueous sodium hydroxide (80.0 g) and then 0.1% hydrochloric acid (35.0 g) were added. The layers were separated, and the organic layer was washed with water (78 mL). The organic layer was filtered to isolate precipitates, which were dried to obtain crystals of Compound 7 (28.90 g, 65.2%).

Compound 3

¹H-NMR (DMSO-d₆) δ: 6.82 (1H, dd, J=7.09, 2.93 Hz), 6.71 (1H, dd, J=11.74, 8.56 Hz), 6.39 (1H, ddd, J=8.56, 3.79, 3.06 Hz), 6.12 (1H, ddd, J=17.18, 10.51, 1.77 Hz), 5.30 (1H, s), 5.15 (1H, dt, J=17.18, 1.77 Hz), 4.95 (1H, dd, J=10.51, 1.71 Hz), 4.82 (2H, bs), 1.50 (3H, d, J=1.10 Hz).

Compound 4

¹H-NMR (DMSO-d₆) δ: 10.55 (1H, s), 7.90-7.97 (2H, m), 7.77-7.86 (2H, m), 7.45 (1H, dd, J=7.21, 2.69 Hz), 6.96-7.04 (2H, m), 6.07 (1H, ddd, J=17.18, 10.51, 1.77 Hz), 5.49 (1H, s), 5.12 (1H, dt, J=17.21, 1.54 Hz), 4.97 (1H, dd, J=10.51, 1.47 Hz), 1.49 (3H, d, J=0.98 Hz).

Compound 7

¹H-NMR (DMSO-d₆) δ: 7.81-7.88 (1H, m), 7.55-7.67 (3H, m), 6.77-6.85 (1H, m), 6.67-6.74 (2H, m), 5.47-5.54 (1H, m), 3.90 (2H, d, J=7.70 Hz), 1.93-1.96 (3H, m).

### Example 1-2

Preparation of Compound 9

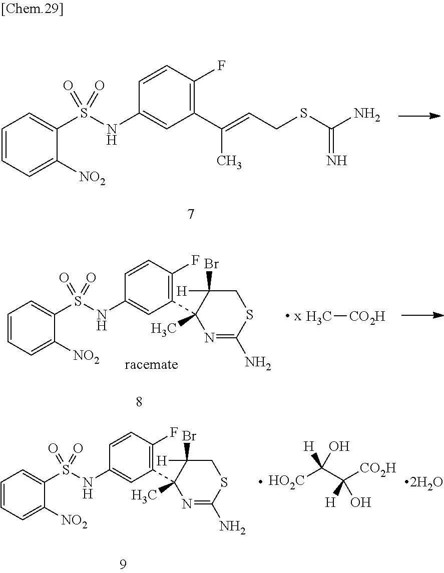

Compound 7 (75.0 g, 176.7 mmol) was dissolved in ethyl acetate (225 mL) and acetic acid (53.1 g, 884.6 mmol). The solution was added to a suspension of N-bromosuccinimide (37.7 g, 211.8 mmol) in ethyl acetate (188 mL), and the mixture was stirred for 2 hours at −20° C. Toluene (300 mL) was added, and the mixture was stirred for 2.2 hours and filtered to obtain 118.9 g of crystals (wet crystals) of Compound 8.

A mixture of the wet crystals of Compound 8 (59.5 g) and (L)-tartaric acid (46.4 g, 309.2 mmol) in water (53 mL), 2-propanol (79 mL) and ethyl acetate (131 mL) (water/2-propanol/ethyl acetate=20/30/50) was stirred for 2 hours at 25° C., filtered and dried to afford 21.67 g of Compound 9 (yield: 35.6%, optical purity: 97.9%).

Compound 9

¹H-NMR (DMSO-d₆) δ: 7.88-7.96 (2H, m), 7.77-7.85 (2H, m), 7.04-7.19 (3H, m), 5.07 (1H, dd, J=4.77, 3.18 Hz), 4.19 (2H, s), 3.23 (1H, dd, J=13.88, 4.95 Hz), 2.88 (1H, dd, J=13.82, 3.06 Hz), 1.59 (3H, s). [α]D+7.3±0.9° (DMSO, 22° C., c=0.518)

### Example 1-3

Preparation of Compound 11

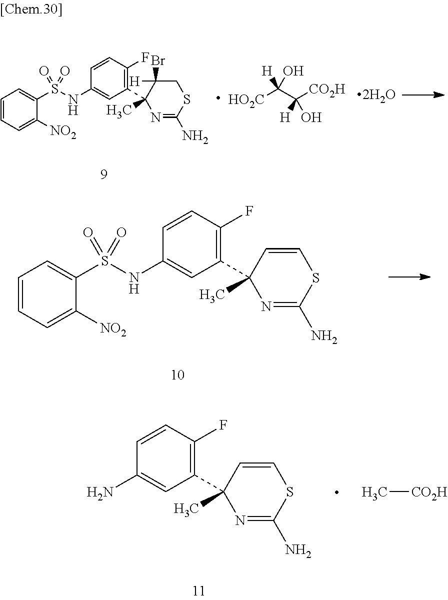

A suspension of Compound 9 (20.0 g, 29.0 mmol) in N,N-dimethylacetamide (30 mL) was cooled to 5° C. 1,8-diazabicyclo(5,4,0)-7-undecene (39.7 g, 260.8 mmol) was added, and the mixture was stirred for 22 hours. Water (70 mL) was added to afford a solution of Compound 10.

To a mixture of ethyl acetate (200 mL), water (40 mL) and 62% sulfuric acid (12.7 g) was added the solution of Compound 10, and the mixture was cooled to 10° C. 15% sulfuric acid (3.7 g) was added, and the mixture was warmed to 20° C. The layers were separated, and the organic layer was washed with 5% sodium chloride in water (95 g). The layers were separated, and the organic layer was concentrated in vacuo to 42 mL. Ethyl acetate (20 mL) and 50% potassium carbonate in water (20 g) were added, and the mixture was warmed to 40° C. 4-chlorobenzenethiol (6.29 g, 43.5 mmol) and ethyl acetate (11 mL) were added, and the mixture was stirred for 1 hour. After cooling to 20° C., ethyl acetate (100 mL), water (68 mL) and 15% hydrochloric acid (42.6 g) were added. The layers were separated, and ethyl acetate (149 mL) and 20% potassium carbonate in water (40.5 g) were added to the aqueous layer. The layers were separated, and the organic layer was washed with water (100 mL). The layers were separated, and the organic layer was concentrated to 20 mL. Acetic acid (1.7 g, 29.0 mmol) was added, and the mixture was cooled to 5° C. and stirred for 90 min, filtered and dried to afford 7.19 g of crystals of Compound 11 (yield: 83.4%, optical purity of (S)-isomer: 100%).

Compound 11

¹H-NMR (DMSO-d₆) δ: 6.74 (1H, dd, J=11.86, 8.56 Hz), 6.62 (1H, dd, J=6.97, 2.93 Hz), 6.35-6.40 (2H, m), 6.11 (1H, dd, J=9.60, 4.71 Hz), 1.90 (3H, s), 1.49 (3H, s). The optical purity was determined as follows.

(Sample Preparation)

25 mg of Compound 11 was weighed and dissolved in a solvent to prepare a 50 mL sample solution.

(Method)

Using liquid chromatography, the peak area was determined by automatic integration method for each of (R)- and (S)-isomers of Compound 11.

(Conditions)

Detector: ultraviolet absorptiometer (wave length: 230 nm)

Column: CHIRALCEL OD-RH, φ4.6×150 mm, 5 μm, (Daicel Corporation)

Column Temp.: constant at around 40° C.

Mobile Phase: water/acetonitrile (LC grade)/methanol (LC grade)/triethylamine (1320:340:340:1)

Flow Rate: 1.0 mL/min (retention time of Compound 11: about 8 min for (R)-isomer, about 9 min for (S)-isomer)

Time span of measurement: over 15 min from the sample injection

Injection Volume: 10 μL

Sample Cooler Temp.: constant at around 25° C.

Autoinjector Rinse Solution: water/acetonitrile (1:1)

### Example 1-4

Preparation of Compound 15

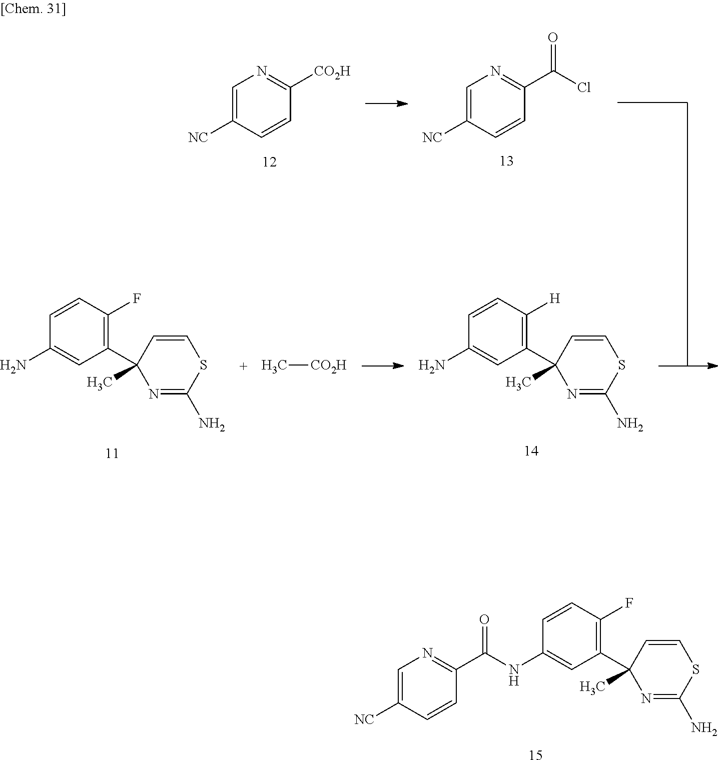

Compound 12 (3.0 g, 20.3 mmol) was dissolved in N-methylpyrrolidone (18 mL), and the solution was cooled to 5° C. Thionyl chloride (3.1 g, 26.1 mmol) was added to obtain a solution of Compound 13.

To a suspension of Compound 11 (5.0 g, 16.8 mmol) in ethyl acetate (50 mL) were added sodium bicarbonate (3.5 g, 42.0 mmol) and water (50 mL), and the mixture was stirred for 5 min at 20° C.

The layers were separated, and the organic layer was concentrated to 10 g under reduced pressure. N-Methylpyrrolidone (5 mL) and 35% hydrochloric acid (0.9 g) were added, and the mixture was cooled to 3° C. The solution of Compound 13 and N-methylpyrrolidone (1.5 mL) were added to obtain a solution of Compound 15.

The solution of Compound 15 was added to a mixture of water (15 mL) and ethyl acetate (10 mL). After stirring the mixture for 1 hour, triethylamine (14.8 g, 14.6 mmol), N-methylpyrrolidone (1.5 mL) and water (5 mL) were added and further stirred for 1 hour. Water (45 mL) was added, and the mixture was stirred for 1 hour, filtered and dried to obtain crystals of Compound 15 (Crystalline Form I, 5.71 g, 92.4%).

Compound 15

¹H-NMR (CDCl₃) δ: 1.71 (3H, s), 4.06 (3H, s), 6.29 (2H, d, J=2.4 Hz), 7.07 (1H, dd, J=11.3, 8.8 Hz), 7.65 (2H, dd, J=6.8, 2.8 Hz), 7.86 (1H, ddd, J=8.8, 4.1, 2.8 Hz), 8.19 (1H, dd, J=8.1, 2.0 Hz), 8.43 (1H, d, J=8.1 Hz), 8.89 (1H, d, J=2.0 Hz), 9.81 (1H, s). [α]D −11.8±1.0° (DMSO, 23° C., c=0.518)

### Example 1-5

To a suspension of Compound 11 (1831 g, 6.2 mol) in ethyl acetate (18 L) were added sodium bicarbonate (1293 g, 15.4 mol) and water (18 L), and the mixture was stirred for 5 min at 20° C. The layers were separated, and the organic layer was concentrated to 3.8 kg under reduced pressure to obtain a concentrated solution of Compound 14.

Compound 12 (912 g, 6.2 mol) was dissolved in N-methylpyrrolidone (64 L), and the solution was cooled to 4° C. Thionyl chloride (951 g, 8.0 mol) was added, and the mixture was stirred for 30 min. The concentrated solution of Compound 14 was added to obtain a solution of Compound 15.

The solution of Compound 15 and N-methylpyrrolidone (1.6 L) were added to water (18 L), and the mixture was stirred for 40 min at 25° C. 24% sodium hydroxide in water (5 kg), sodium bicarbonate (259 g, 3.1 mmol) and water (2.7 L) were added to the mixture. The mixture was stirred for 1 hour, filtered and dried to obtain crystals (metastable Form II) of Compound 15 (1.93 kg, 85.4%).

### Example 1-6

When the reaction solution was neutralized with an inorganic base such as sodium hydroxide (e.g. Example 1-5), precipitates appeared during neutralization. The obtained precipitates were a metastable crystal form II or mixture of a stable crystal form I and a metastable crystal form II. The size of the obtained crystals was small and the filtration speed was slow.

On the other hand, when the reaction solution was neutralized with triethylamine, such as in Example 1-4, or iPr₂NEt, the precipitates did not appeared during neutralization. The precipitates appeared during the subsequent addition of water. The obtained precipitates were the crystal form I. The size of the obtained crystals was larger and the filtration speed was faster than that by using an inorganic base. The results are shown below.

TABLE 1

| | Method A | Method B |
|---|---|---|
| Example | Example 1-4 | Example 1-5 |
| Crystallization | Anti-solvent | Neutralization |
| Method | Crystallization | Crystallization |

| | | | | NH₃/ | Aqueous |
|---|---|---|---|---|---|
| Base | Triethylamine | iPr₂NEt | Pyridine | 2-propanol | NaOH |
| Method | A | A | A* | B | B |
| Crystal | I | I | I | I | II or |
| Form | | | | | I + II |
| Crystal | Large | Large | Small | Small | Small |
| Size | | | | | |
| residual | 0 ppm | N.A. | N.A. | N.A. | Up to |
| Solvent | | | | | 800 ppm |
| (N-methyl | | | | | |
| pyrrolidone) | | | | | |

*Since basicity was weak and neutralization was not completed, an aqueous solution of sodium hydroxide was also used.
N.A.: Not applicable

### Example 2

Evaluation of Acid for Diastereomeric Salt Formation

1) Method

a. The following compound (a mixture of the (4R,5R)- and (4S,5S)-isomers) was dissolved in THF/DMF (9/1), and the solution was dispensed to a 2 mL 96-well deep-well plate (10 mg/well). Then, the acid solution listed below (1.05 eq. of the compound) was added to each well.

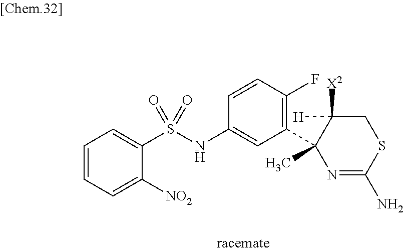

b. After evaporation of the solvent, zirconia balls (3 mm diameter) and 200 μL of the solvent were added, and the plate was sealed.

c. The plate was shaken in a plate shaker (1000 rpm) for 1 hr at 15° C. and allowed to stand overnight at 3° C.

d. To the sample with no precipitation observed was added ethyl acetate (200 μL), and the plate was shaken (1500 rpm) for 1 hr at 15° C.

e. The supernatant was taken into a 2 mL 96-well deep-well filter plate and centrifugally filtered.

f. The filtrate was diluted with MeCN/H₂O (8/2) and analyzed by HPLC under the following conditions.

2) HPLC Conditions

Instrument: SHIMADZU Prominence UFLC 20A Series

Column: CHIRALPAK AS-RH, 5 μm, 4.6 mm I.D.×150 mm (Daicel Corporation)

Mobile Phase: 10 mM NH₄HCO₃ aq./MeCN=60/40 isocratic

Flow Rate: 1.0 mL/min

Wavelength: 240 nm

Injection Volume: 5 μL

Column Temp.: 25° C.

tR: 6.5 min, 7.4 min

3) Acid Solution

L-(+)-Tartaric Acid: 0.5 mol/L in water

D-(−)-Tartaric Acid: 0.5 mol/L in water

(−)-Dibenzoyl-L-Tartaric Acid: 0.5 mol/L in MeOH/water (95/5)

(+)-Dibenzoyl-D-Tartaric Acid: 0.5 mol/L in MeOH/water (95/5)

Di-P-Toluoyl-L-Tartaric Acid: 0.5 mol/L in MeOH/water (95/5)

Di-P-Toluoyl-D-Tartaric Acid: 0.5 mol/L in MeOH/water (95/5)

L-(−)-Malic Acid: 0.5 mol/L in water

D-(+)-Malic Acid: 0.5 mol/L in water

(−)-10-Camphorsulfonic Acid: 0.5 mol/L in water

(+)-10-Camphorsulfonic Acid: 0.5 mol/L in water

D-(+)-Camphoric Acid: 0.5 mol/L in MeOH/water (50/50)

L-Pyroglutamic acid: 0.5 mol/L in water

L-(+)-Mandelic Acid: 0.5 mol/L in MeOH/water (50/50)

D-(−)-Mandelic Acid: 0.5 mol/L in MeOH/water (50/50)

Naproxen: 0.5 mol/L in THF/water (95/5)

D-(−)-Quinic Acid: 0.5 mol/L in water

4) Results

The results are shown below. The diastereomer of interest (the 4R,5R-isomer, retention time: 6.5 min) was significantly less in the supernatant from the sample added with (L)-tartaric acid or (D)-malic acid, indicating that the desired 4R,5R-diastereomeric salt was specifically obtained in the precipitation.

TABLE 2-1

| | | | Peak area % | |
|---|---|---|---|---|
| | | | tR = | tR = |
| solvent | Acid | Observation | 6.5 min | 7.4 min |
| 95% | L-(+)-Tartaric Acid | *1 | 21 | 79 |
| MeCN in | D-(−)-Tartaric Acid | *1 | 73 | 27 |
| water | (−)-Dibenzoyl-L-Tartaric | X | 50 | 50 |
| | Acid | | | |
| | (+)-Dibenzoyl-D-Tartaric | X | 50 | 50 |
| | Acid | | | |
| | Di-P-Toluoyl-L-Tartaric | X | 50 | 50 |
| | Acid | | | |
| | Di-P-Toluoyl-D-Tartaric | X | 50 | 50 |
| | Acid | | | |
| | L-(−)-Malic Acid | X | 50 | 50 |
| | D-(+)-Malic Acid | X | 50 | 50 |
| | (−)-10-Camphorsulfonic | X | 50 | 50 |
| | Acid | | | |
| | (+)-10-Camphorsulfonic | X | 50 | 50 |
| | Acid | | | |
| | D-(+)-Camphoric Acid | X | 50 | 50 |
| | L-Pyroglutamic acid | *2 | 50 | 50 |
| | L-(+)-Mandelic Acid | *2 | 50 | 50 |
| | D-(−)-Mandelic Acid | *3 | 50 | 50 |
| | Naproxen | X | 50 | 50 |
| | D-(−)-Quinic Acid | *3 | 50 | 50 |
| TABLE 2-2 | | | | |
| 95% | L-(+)-Tartaric Acid | X | 50 | 50 |
| 2-BuOH | D-(−)-Tartaric Acid | X | 50 | 50 |
| in | (−)-Dibenzoyl-L-Tartaric | X | 50 | 50 |
| water | Acid | | | |
| | (+)-Dibenzoyl-D-Tartaric | X | 50 | 50 |
| | Acid | | | |
| | Di-P-Toluoyl-L-Tartaric | X | 50 | 50 |
| | Acid | | | |
| | Di-P-Toluoyl-D-Tartaric | X | 50 | 50 |
| | Acid | | | |
| | L-(−)-Malic Acid | X | 50 | 50 |
| | D-(+)-Malic Acid | *1 | 12 | 88 |
| | (−)-10-Camphorsulfonic | X | 50 | 50 |
| | Acid | | | |
| | (+)-10-Camphorsulfonic | X | 50 | 50 |
| | Acid | | | |
| | D-(+)-Camphoric Acid | *1 | 50 | 50 |
| | L-Pyroglutamic acid | X | 50 | 50 |
| | L-(+)-Mandelic Acid | *3 | 50 | 50 |
| | D-(−)-Mandelic Acid | X | 50 | 50 |
| | Naprozen | X | 50 | 50 |
| | D-(−)-Quinic Acid | *3 | 50 | 50 |

*1: Precipitation was observed after addition of EtOAc and shaking for 1 hr at 15° C.
*2: Precipitation was observed after addition of solvent and shaking for 1 hr at 15° C.
*3: Precipitation was observed after standing overnight at 3° C.
X: No precipitation was observed.

### Example 3-1

Evaluation of Solvent for Diastereomeric Salt Formation

1) Method

a. The following compound (tartrate salt dihydrate) was placed in a vial (10 or 100 mg for each vial).

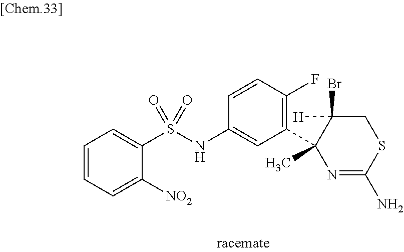

b. The solvent listed below (500 μL or 1 mL) was added to the vial.

c. The vial was shaken in a rotating shaker for 1 hr at 25° C. and followed by filtration through PTFE filter.

d. The filtrate was added to a 96-well HPLC plate and diluted with MeCN/water (55/45).

e. The samples were checked for any air bubble or precipitation, the plate was sealed and shaken in a plate mixer.

f. The sample was analyzed by HPLC under the following conditions.

Column: CHIRALCEL OZ-RH 4.6 mm×150 mm 5 μm (Daicel Corporation)

Mobile Phase: 20 mM HCO₂NH₄ H₂O/MeCN isocratic

Organic Solvent Ratio: 55%

Flow Rate: 1.0 mL/min

Column Temp.: 25° C.

Wavelength: 256 nm

Injection Volume: 5 μL

tR: 3.52 min, 4.27 min

g. The diastereomer excess (de %) of the 4R,5R-isomer in the precipitate was calculated from the contents of the 4R,5R-isomer and the 4S,5S-isomer found in the supernatant.

2) Results

The diastereomer excess (de %) of the 4R,5R-isomer is shown below. The efficiency of optical resolution was improved by using water in combination with an organic solvent, such as acetonitrile, methanol, 2-propanol, butanol, ethyl acetate, ethyl formate, acetone, and methyl ethyl ketone.

TABLE 3

| | Water content | | | | | | | | | | |
|---|---|---|---|---|---|---|---|---|---|---|---|
| solvent | 0% | 10% | 20% | 25% | 30% | 40% | 50% | 60% | 75% | 80% | 100% |
| CH₃CN | 3.6 | 54.4 | | 39.5 | | 15.7 | 46.8 | 19.5 | 57.1 | | 29.7 |
| MeOH | 5.0 | | | | | | 6.0 | | | 61.8 | 29.7 |
| 2-propanol | 4.0 | 31.7 | 44.4 | | 44.7 | 45.5 | 18.3 | | 21.9 | | 29.7 |
| 1-Butanol | 10.3 | | | 50.6 | | | 34.8 | | 51.6 | | 29.7 |
| EtOAc | 4.2 | | 35.0 | | | | 18.8 | | 9.6 | | 29.7 |
| Ethyl formate | 4.6 | 51.9 | 56.4 | 54.7 | 58.0 | 56.5 | 64.4 | | 51.2 | | 29.7 |
| Acetone | 0.3 | | | | | | 3.7 | | | 67.4 | 29.7 |
| Methyl ethyl ketone | 4.8 | 47.1 | | 42.3 | | 18.4 | 52.9 | 20.8 | 52.2 | | 29.7 |
| Water | 29.7 | | | | | | | | | | |

### Example 3-2

Diastereomeric salt formation was evaluated using water/2-propanol/ethyl acetate as a solvent.

1) Method

a. The tartrate salt dihydrate of the following compound was placed in a vial (100 mg for each vial).

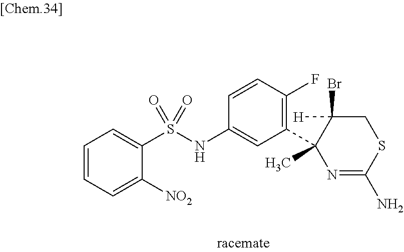

b. 250 μL of water/2-propanol/ethyl acetate (20/40/40 (v/v)) was added to the vial.

c. The vial was shaken in a rotating shaker for 1 hr at 25° C. and followed by filtration through PTFE filter.

d. The filtrate was added to a 96-well HPLC plate and diluted to 200 times with MeCN/water (55/45).

e. The samples were checked for any air bubble or precipitation, and the plate was sealed and shaken in a plate mixer.

f. The sample was analyzed by HPLC under the following conditions.

Column: CHIRALCEL OZ-RH 4.6 mm×150 mm 5 μm (Daicel Corporation)

Mobile Phase: 20 mM HCO₂NH₄ H₂O/MeCN isocratic

Organic Solvent Ratio: 55%

Flow Rate: 1.0 mL/min

Column Temp.: 25° C.

Wavelength: 256 nm

Injection Volume: 5 μL

3) Results

HPLC analysis revealed that the concentration of the 4R,5R- and 4S,5S-diastereomers in the filtrate was 19 mg/mL and 149 mg/mL, respectively, indicating that the desired 4R,5R-diastereomer salt was obtained specifically. The diastereomer excess (de %) of the 4R,5R-diastereomer of the obtained salt was 77%.

### Example 4

Different acids and solvents were tested for crystallization of the Compound 14.

1) Method

The Compound 14 (10 mg or 100 mg) was dissolved in a solvent. Then, an acid (sulfuric acid, hydrochloric acid, hydrobromic acid, acetic acid, formic acid or phosphoric acid) was added, and the mixture was stirred for one day at room temperature.

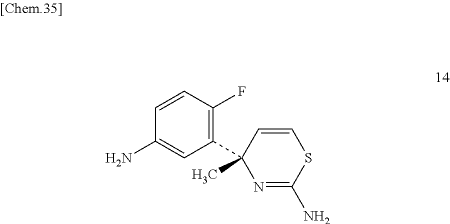

For the samples observed to be crystallized, HPLC analysis was carried out under the following conditions.

Column: Unison UK-C18, 3 μm, 4.6 mm I.D.×150 mm

Flow Rate: 1.0 mL/min

Wavelength: 254 nm

Mobile phase A: 0.1% Trifluoroacetic acid/Water

Mobile phase B: 0.1% Trifluoroacetic acid/acetonitrile

Flowing of the mobile phase: Control the gradient by mixing the mobile phase A and B as directed in Table 4.

TABLE 4

| Time after injection | Mobile phase | Mobile phase |
|---|---|---|
| of sample (min) | A (%) | B (%) |
| 0 to 15 | 95 to 5 | 5 to 95 |
| 15 to 15.01 | 5 to 95 | 95 to 5 |
| 15.01 to 20 | 95 | 5 |

Column Temp.: 35° C.

2) Results

The results are shown in Table 5. In 10 mg scale, the Compound 14 formed crystals of sulfate salt with sulfuric acid in water. Also, crystals of acetate salt were formed with acetic acid in 2-propanol, acetonitrile or ethyl acetate.

In 100 mg scale, sulfuric acid and acetic acid were tested. The Compound 14 was crystallized in 70% yield in water with sulfuric acid while 91% yield in ethyl acetate with acetic acid (the yields were calculated as 0.5 sulfate salt and mono acetate salt).

The HPLC analysis showed that sulfate salt crystals of the Compound 14 occupied 97.8 area % and the Compound 14 in the mother liquid occupied 99.5 area %. On the other hand, acetate salt crystals of the Compound 14 occupied 98.7 area % while the Compound 14 in the mother liquid occupied 80.8 area %.

An effect on purification of the Compound 14 was found by crystallization of acetate salt but not by crystallization of sulfate salt.

TABLE 5

| | Scale | | | | | |
|---|---|---|---|---|---|---|
| No. | (mg) | Solvent, mL | | Acid equivalent | | observation |
| 1 | 10 | water | 0.2 | 1M sulfuric | 0.6 | Crystallized |
| | | | | acid aq | | |
| 2 | 10 | methanol | 0.1 | 1M sulfuric | 0.6 | Not |
| | | | | acid aq | | crystallized |
| 3 | 10 | ethanol | 0.1 | 1M sulfuric | 0.6 | Not |
| | | | | acid aq | | crystallized |
| 4 | 10 | 2-propanol | 0.1 | 1M sulfuric | 0.6 | Not |
| | | | | acid aq | | crystallized |
| 5 | 10 | acetone | 0.1 | 1M sulfuric | 0.6 | Not |
| | | | | acid aq | | crystallized |
| 6 | 10 | acetonitrile | 0.1 | 1M sulfuric | 0.6 | Not |
| | | | | acid aq | | crystallized |
| 7 | 10 | methanol | 0.1 | 1M | 2.4 | Not |
| | | | | hydrochloric | | crystallized |
| | | | | acid/Dioxane | | |
| 8 | 10 | ethanol | 0.1 | 1M | 2.4 | Not |
| | | | | hydrochloric | | crystallized |
| | | | | acid/Dioxane | | |
| 9 | 10 | 2-propanol | 0.2 | 1M | 2.4 | Not |
| | | | | hydrochloric | | crystallized |
| | | | | acid/Dioxane | | |
| 10 | 10 | acetone | 0.5 | 1M | 2.4 | Not |
| | | | | hydrochloric | | crystallized |
| | | | | acid/Dioxane | | |
| 11 | 10 | acetonitrile | 0.5 | 1M | 2.4 | Not |
| | | | | hydrochloric | | crystallized |
| | | | | acid/Dioxane | | |
| 12 | 10 | ethanol | 0.1 | 1M | 2.4 | Not |
| | | | | hydrochloric | | crystallized |
| | | | | acid/Dioxane | | |
| 13 | 10 | 2-propanol | 0.5 | 1M | 2.4 | Not |
| | | | | hydrochloric | | crystallized |
| | | | | acid/Dioxane | | |
| 14 | 10 | acetonitrile | 0.5 | 1M | 2.4 | Not |
| | | | | hydrochloric | | crystallized |
| | | | | acid/Dioxane | | |
| 15 | 10 | methanol | 0.2 | phosphoric | 4 | Not |
| | | | | acid | | crystallized |
| 16 | 10 | ethanol | 0.5 | phosphoric | 4 | Precipitated |
| | | | | acid | | but |
| | | | | | | transformed |
| | | | | | | into |
| | | | | | | candy-like |
| | | | | | | material |
| | | | | | | during |
| | | | | | | filtration |
| 17 | 10 | methanol | 0.1 | acetic acid | 4 | Not |
| | | | | | | crystallized |
| 18 | 10 | acetic acid | 0.1 | acetic acid | 40 | Not |
| | | | | | | crystallized |
| 19 | 10 | 2-propanol | 0.2 | acetic acid | 4 | Crystallized |
| 20 | 10 | acetonitrile | 0.2 | acetic acid | 4 | Crystallized |
| 21 | 10 | EtOAc | 0.2 | acetic acid | 4 | Crystallized |
| 22 | 10 | methanol | 0.2 | formic acid | 6 | Not |
| | | | | | | crystallized |
| 23 | 10 | ethanol | 0.2 | formic acid | 6 | Not |
| | | | | | | crystallized |
| 24 | 10 | 2-propanol | 0.2 | formic acid | 6 | Not |
| | | | | | | crystallized |
| 25 | 10 | acetonitrile | 0.2 | formic acid | 6 | Not |
| | | | | | | crystallized |
| 26 | 10 | acetone | 0.2 | formic acid | 6 | Not |
| | | | | | | crystallized |
| 27 | 10 | EtOAc | 0.2 | formic acid | 6 | Not |
| | | | | | | crystallized |
| 28 | 100 | water | 0.2 | 1M sulfuric | 0.5 | Crystallized |
| | | | | acid aq | | |
| 29 | 100 | EtOAc | 1 | acetic acid | 1.2 | Crystallized |
| | | | | | | (effective in |
| | | | | | | purification) |

### Example 5

X-Ray Powder Diffraction Analysis

According to “X-Ray Powder Diffraction Method” described in Japanese Pharmacopoeia, the X-ray powder diffraction pattern for the crystal of Compound 9 obtained in Example 1-2 was acquired on Bruker D8 Discover diffractometer (Cu Kα radiation, 40 kV, 40 mA, detection in reflection mode, incident angle 3° and 12°).

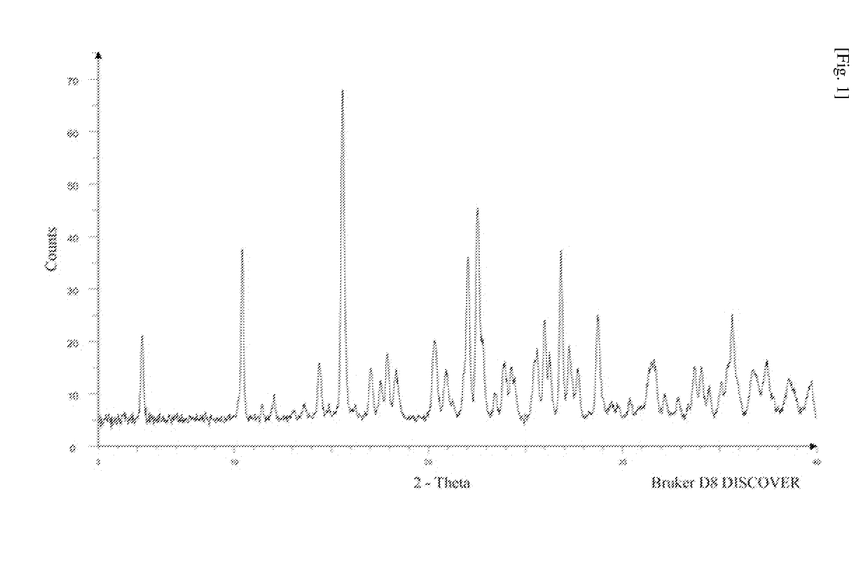

The X-ray powder diffraction pattern is shown in FIG. 1.

### Example 6

Thermal Analysis

3.67 mg of the crystal of Compound 9 was massed out in a non-sealed aluminum pan. The thermal behavior of the crystal was observed by TG/DTA analysis under the following condition.

Instrument: TG/DTA6300 (Hitachi High-Tech Science Corporation)

Measurement range: room temperature to 350° C.

Heating rate: 10° C./min

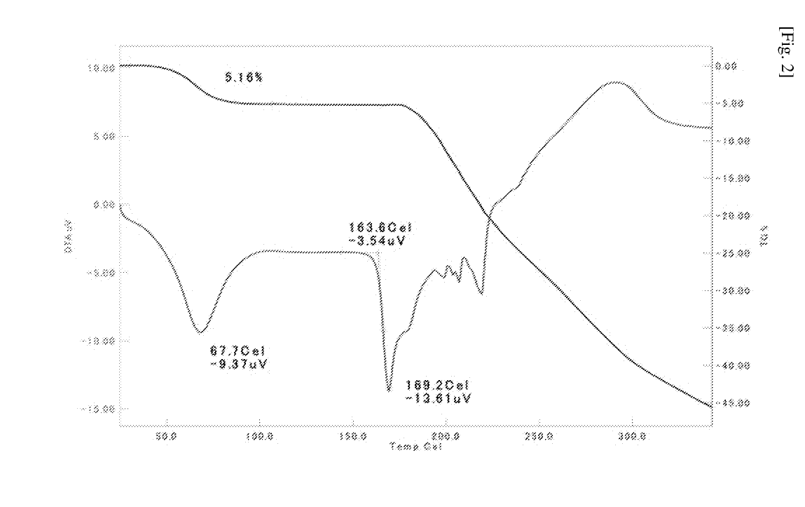

The result is shown in FIG. 2. FIG. 2 shows 5.16% weight loss on heating up to 200° C., indicating that the crystal is dihydrate crystal (dihydrate theory=5.22%).

### Example 7

Dynamic Vapor Sorption (DVS)

15.25 mg of the crystal of Compound 9 was massed out in a sample pan. The water sorption in the crystal was observed by dynamic vapor sorption analysis under the following condition.

Instrument: IGA SORP (Hiden Isochema)

Measurement point: 5% (in fact, from 5.6%) to 95% relative humidity (RH) at 5% intervals, then 95% to 5% (in fact, to 7.9%) at 5% intervals

Temperature: 25° C.

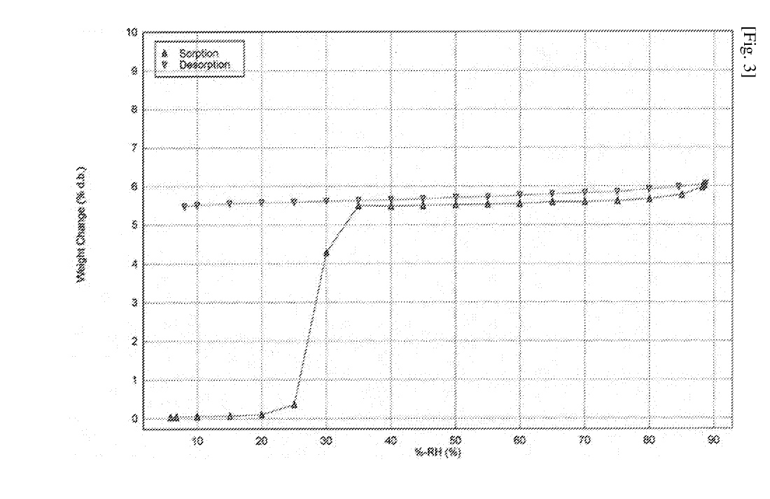

The result is shown in FIG. 3. FIG. 3 shows slightly more than 5% of water at ambient temperature, indicating that the crystal is dihydrate crystal.

### Example 8

X-Ray Powder Diffraction Analysis of Compound 11

X-ray powder diffraction patterns for the crystal of Compound 11 obtained in Example 1-3 were acquired on RINT-TTRIII (Rigaku) with Cu Kα radiation (parallel beam), according to the method described in Japanese Pharmacopoeia under the following condition.

Current for the X-ray vacuum tube: 300 mA

Voltage for the X-ray vacuum tube: 50 Kv

Sample holder: aluminum

Scan range (θ):4° to 40°

Sampling angle: 0.020°

Scan speed: 5°/min

Divergence slit: 1.00 mm

Divergence vertical slit: 10 mm

Scatter slit: 1 mm

Receiving slit: open

Parallel slit: 100 mm

Spin rate of sample holder: 120 rpm

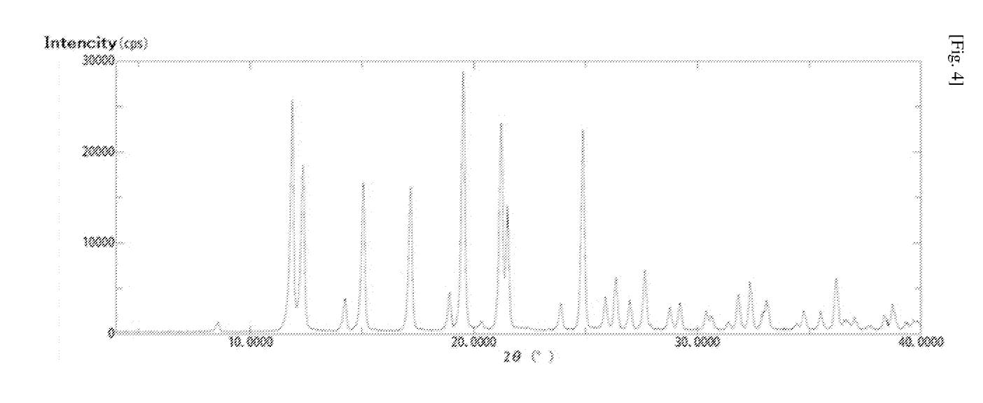

The results are shown in FIG. 4 and Table 6.

TABLE 6
2θ (°)
11.9
12.3
15.0
17.2
19.5
21.2
21.5
24.9
27.6
36.2

In the X-ray powder diffraction pattern, the peaks at 11.9±0.2° 2θ, 12.3±0.2° 2θ, 15.0±0.2° 2θ, 17.2±0.2° 2θ, 19.5±0.2° 2θ, 21.2±0.2° 2θ, 21.5±0.2° 2θ, 24.9±0.2° 2θ, 27.6±0.2° 2θ, and 36.2±0.2° 2θ, in particular, 11.9±0.2° 2θ, 12.3±0.2° 2θ, 19.5±0.2° 20, 21.2±0.2° 2θ, and 24.9±0.2° 2θ are the main features of the pattern.

### Example 9

X-Ray Powder Diffraction Analysis of Compound 15

1) Method

X-ray powder diffraction patterns for the crystal of Compound 15 (stable crystalline Form I obtained in Example 1-4, and metastable Form II obtained in Example 1-5) were acquired in the same manner as described in Example 8.

2) Results

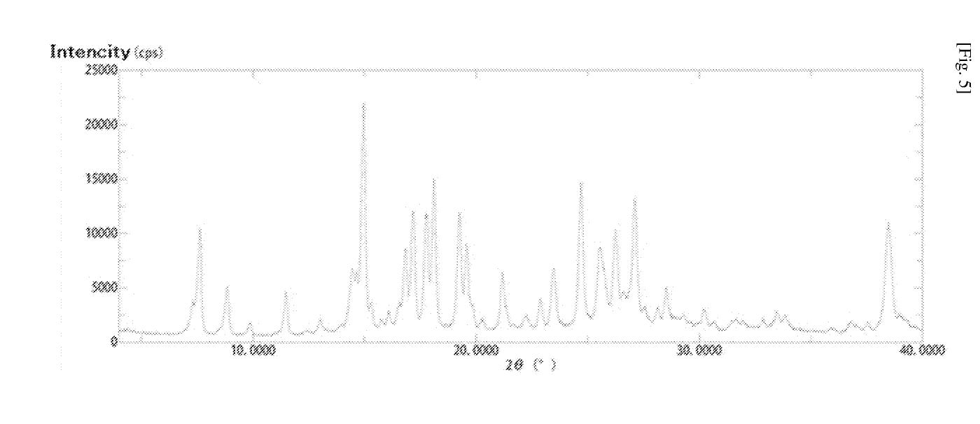

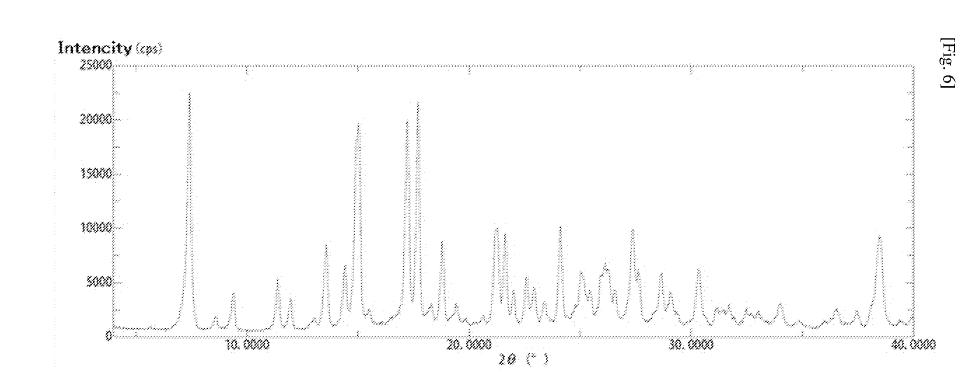

X-ray powder diffraction patterns are shown in FIG. 5 and Table 7 (stable Form I) and FIG. 6 and Table 8 (metastable Form II). The peak at around 38° 2θ is of aluminum from the sample holder.

TABLE 7
2θ (°)

7.6
8.8
11.5
15.0
17.2
18.1
19.2
24.7
26.2
27.1

In the X-ray powder diffraction pattern in FIG. 5 (stable Form I), the peaks at 7.6±0.2° 2θ, 8.8±0.2° 2θ, 11.5±0.2° 2θ, 15.0±0.2° 2θ, 17.2±0.2° 2θ, 18.1±0.2° 2θ, 19.2±0.2° 2θ, 24.7±0.2° 2θ, 26.2±0.2° and 27.1±0.2° 2θ, in particular, 7.6±0.2° 2θ, 15.0±0.2° 2θ, 18.1±0.2° 2θ, 24.7±0.2° and 27.1±0.2° are the main features of the pattern.

TABLE 8
2θ (°)

7.4
9.4
13.6
15.0
17.2
17.7
18.8
21.6
24.1
27.4

In the X-ray powder diffraction pattern in FIG. 6 (metastable Form II), the peaks at 7.4±0.2° 2θ, 9.4±0.2° 2θ, 13.6±0.2° 2θ, 15.0±0.2° 2θ, 17.2±0.2° 2θ, 17.7±0.2° 2θ, 18.8±0.2° 2θ, 21.6±0.2° 2θ, 24.1±0.2° and 27.4±0.2° 2θ, in particular, 7.4±0.2° 2θ, 15.0±0.2° 2θ, 17.2±0.2° 2θ, 17.7±0.2° and 27.4±0.2° are the main features of the pattern.

### Example 9-2

Single Crystal Structure Analysis

0.1 g of Compound 15 was added to 10 mL of acetonitrile, and dissolved at 60° C. The solution was allowed to stand at room temperature for 2 days, and single crystals of Compounds 15 were recrystallized.

X-ray diffraction intensities for the single crystal Form I of Compound 15 were collected on a Rigaku R-AXIS RAPID imaging plate area detector with graphite monochromated Cu Kα radiation (X=1.54187A) at −100.0° C. Data collection and reduction were performed using RAPID-AUTO (RAPID-AUTO. Rigaku Corporation, 2006). Data were corrected by the Lorentz polarization and absorption factors.

The crystal structure was solved by the direct-method program SHELXS97 (Sheldrick, G. M. (2008), Acta Cryst. A64, 112-122), and refined using SHELXL97 (Sheldrick, G. M. (2008), Acta Cryst. A64, 112-122) with full-matrix least squares and anisotropic temperature factors for all non-hydrogen atoms. The hydrogen atoms were located by calculation and refined as riding model using the default parameter of SHELXL97. R1 (I>2.00s(I)) was 0.0680, and no missing or misplaced electron density observed in the final difference Fourier.

The asymmetric unit contains two Compound 15 molecules, which are herein after referred to as “Molecule I” and “Molecule II”. The absolute configuration of the molecule was based on using Flack Parameter (Flack, H. D. (1983), Acta Cryst. A39, 876-881). The Flack parameter (x) was determined to be 0.05(2), and thus, the absolute configuration of Molecule I and Molecule II were both confirmed as S configuration.

Crystal data and data collection parameters of X-ray diffraction analysis are shown in Table 9. Atomic coordinates of non-hydrogen atom and hydrogen atom are shown in Table 10 (non-hydrogen atom) and Table 11 (hydrogen atom).

TABLE 9

| space group | P2₁2₁2 |
|---|---|
| a (Å) | 7.13206 (10) |
| b (Å) | 39.5348 (7) |
| c (Å) | 12.0611 (2) |
| α (°) | 90 |
| β (°) | 90 |
| γ (°) | 90 |
| V(Å³) | 3400.81 (10) |
| Z | 8 |
| d_{calc} (g/cm³) | 1.435 |
| temperature (° C.) | −100 |

TABLE 10-1

| atom | x | y | z | B_{eq} |
|---|---|---|---|---|
| S(1) | −0.0820(2) | 0.67975(4) | 0.43614(13) | 5.82(3) |
| S(2) | 0.7781(2) | 0.74368(4) | 0.12771(13) | 6.13(3) |
| F(1) | 0.4421(5) | 0.65261(8) | 0.7399(2) | 6.21(7) |
| F(2) | 0.7791(4) | 0.65039(8) | −0.1304(2) | 6.34(7) |
| O(1) | 0.6746(6) | 0.53562(9) | 0.3864(3) | 5.77(8) |
| O(2) | 1.1147(5) | 0.55187(10) | 0.2375(3) | 5.62(8) |
| N(1) | 0.1274(7) | 0.69741(12) | 0.2677(3) | 5.82(10) |
| N(2) | 0.2887(6) | 0.69814(10) | 0.4321(3) | 4.78(8) |
| N(3) | 0.5324(6) | 0.58599(10) | 0.3431(3) | 4.72(8) |
| N(4) | 0.5387(6) | 0.57043(11) | 0.1273(3) | 4.73(8) |
| N(5) | 0.6859(8) | 0.53279(13) | −0.2380(4) | 6.51(11) |
| N(6) | 0.5659(7) | 0.73213(12) | 0.2990(3) | 5.73(10) |
| N(7) | 0.5207(6) | 0.69332(11) | 0.1607(3) | 4.82(8) |
| N(8) | 0.9818(6) | 0.60237(10) | 0.2831(3) | 4.91(8) |
| N(9) | 1.0338(6) | 0.59054(11) | 0.4996(3) | 4.88(8) |
| N(10) | 1.1785(8) | 0.54851(13) | 0.8593(4) | 6.45(11) |
| C(1) | 0.4570(8) | 0.71428(14) | 0.5941(4) | 5.58(11) |
| C(2) | 0.3211(7) | 0.68752(13) | 0.5466(4) | 4.77(9) |
| C(3) | 0.1472(7) | 0.68547(14) | 0.6163(4) | 5.10(10) |
| C(4) | −0.0257(8) | 0.68167(14) | 0.5774(4) | 5.36(11) |
| C(5) | 0.1325(7) | 0.69298(13) | 0.3795(4) | 5.14(10) |
| C(6) | 0.4134(7) | 0.65229(12) | 0.5446(4) | 4.57(9) |
| C(7) | 0.4387(6) | 0.63481(11) | 0.4466(3) | 4.25(8) |
| C(8) | 0.5166(7) | 0.60172(13) | 0.4469(4) | 4.51(9) |
| C(9) | 0.5710(7) | 0.58706(13) | 0.5453(4) | 4.70(9) |
| C(10) | 0.5471(7) | 0.60482(13) | 0.6432(4) | 5.28(11) |
| C(11) | 0.4683(7) | 0.63645(13) | 0.6407(4) | 4.82(10) |
| C(12) | 0.6121(7) | 0.55499(13) | 0.3184(4) | 4.80(10) |
| C(13) | 0.6207(7) | 0.54788(12) | 0.1961(4) | 4.52(9) |
| C(14) | 0.5545(7) | 0.56510(13) | 0.0194(4) | 5.27(11) |
| C(15) | 0.6557(8) | 0.53726(13) | −0.0248(4) | 5.01(10) |
| C(16) | 0.6722(8) | 0.53453(14) | −0.1435(5) | 5.54(11) |
| C(17) | 0.7355(7) | 0.51465(13) | 0.0480(4) | 4.96(10) |
| C(18) | 0.7200(7) | 0.51997(13) | 0.1605(4) | 5.01(10) |
| C(19) | 0.4016(7) | 0.65822(14) | 0.0148(4) | 5.48(11) |
| C(20) | 0.5749(7) | 0.67806(13) | 0.0528(4) | 4.88(9) |
| C(21) | 0.6238(8) | 0.70498(14) | −0.0323(4) | 5.41(11) |
| C(22) | 0.7154(9) | 0.73346(15) | −0.0075(4) | 6.04(12) |
| C(23) | 0.6044(7) | 0.71993(14) | 0.1979(4) | 5.02(10) |
| C(24) | 0.7416(7) | 0.65366(12) | 0.0659(4) | 4.31(9) |
| C(25) | 0.7964(7) | 0.64016(12) | 0.1678(4) | 4.54(9) |
| C(26) | 0.9344(7) | 0.61519(12) | 0.1756(4) | 4.42(9) |
| C(27) | 1.0175(8) | 0.60300(13) | 0.0831(4) | 4.92(10) |
| C(28) | 0.9674(8) | 0.61549(13) | −0.0216(4) | 5.27(11) |
| C(29) | 0.8295(8) | 0.63972(13) | −0.0270(4) | 5.07(10) |
| C(30) | 1.0648(7) | 0.57258(14) | 0.3074(4) | 4.82(10) |
| C(31) | 1.0876(7) | 0.56633(13) | 0.4297(4) | 4.80(10) |
| C(32) | 1.0541(7) | 0.58506(14) | 0.6063(4) | 5.21(11) |
| C(33) | 1.1337(8) | 0.55493(14) | 0.6484(4) | 5.17(10) |
| C(34) | 1.1582(8) | 0.55139(14) | 0.7669(5) | 5.65(12) |
| C(35) | 1.1901(8) | 0.53009(14) | 0.5745(4) | 5.12(10) |
| C(36) | 1.1648(8) | 0.53521(13) | 0.4622(4) | 5.06(10) |

B_{eq} = 8/3 π²(U₁₁(aa*)² + U₂₂(bb*)² + U₃₃(cc*)² + 2U₁₂(aa*bb*)cos γ + 2U₁₃(aa*cc*)cos β + 2U₂₃(bb*cc*)cos α)

TABLE 11

| atom | x | y | z | Bᵢₛₒ |
|---|---|---|---|---|
| H(1) | 0.5069 | 0.7064 | 0.6652 | 6.70 |
| H(2) | 0.3898 | 0.7356 | 0.6053 | 6.70 |
| H(3) | 0.5606 | 0.7178 | 0.5420 | 6.70 |
| H(4) | 0.1619 | 0.6870 | 0.6944 | 6.12 |
| H(5) | −0.1252 | 0.6799 | 0.6294 | 6.43 |
| H(6) | 0.2292 | 0.7037 | 0.2320 | 6.98 |
| H(7) | 0.0224 | 0.6940 | 0.2312 | 6.98 |
| H(8) | 0.4037 | 0.6450 | 0.3783 | 5.10 |
| H(9) | 0.6244 | 0.5651 | 0.5460 | 5.64 |
| H(10) | 0.5850 | 0.5951 | 0.7118 | 6.33 |
| H(11) | 0.4859 | 0.5973 | 0.2865 | 5.67 |
| H(12) | 0.4961 | 0.5805 | −0.0304 | 6.33 |
| H(13) | 0.8010 | 0.4955 | 0.0208 | 5.95 |
| H(14) | 0.7760 | 0.5049 | 0.2121 | 6.01 |
| H(15) | 0.3590 | 0.6434 | 0.0749 | 6.58 |
| H(16) | 0.4338 | 0.6445 | −0.0502 | 6.58 |
| H(17) | 0.3012 | 0.6741 | −0.0047 | 6.58 |
| H(18) | 0.5874 | 0.7014 | −0.1072 | 6.49 |
| H(19) | 0.7473 | 0.7486 | −0.0656 | 7.25 |
| H(20) | 0.4824 | 0.7220 | 0.3411 | 6.88 |
| H(21) | 0.6242 | 0.7503 | 0.3233 | 6.88 |
| H(22) | 0.7383 | 0.6482 | 0.2336 | 5.45 |
| H(23) | 1.1104 | 0.5859 | 0.0890 | 5.91 |
| H(24) | 1.0271 | 0.6074 | −0.0869 | 6.32 |
| H(25) | 0.9536 | 0.6154 | 0.3398 | 5.90 |
| H(26) | 1.0138 | 0.6019 | 0.6570 | 6.25 |
| H(27) | 1.2455 | 0.5098 | 0.6008 | 6.14 |
| H(28) | 1.1982 | 0.5184 | 0.4093 | 6.07 |

Bᵢₛₒ = isotropic temperature factor

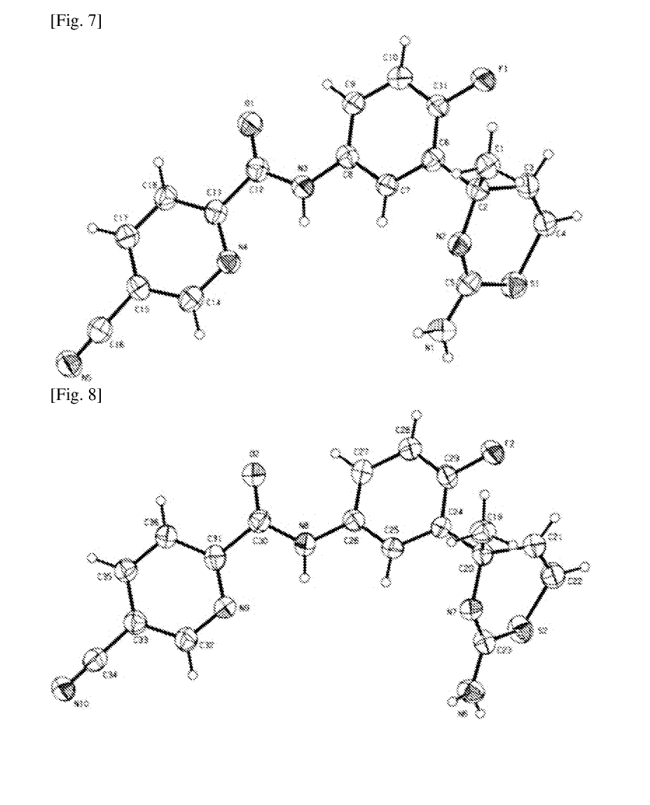

Displacement ellipsoid plots of Molecule I and Molecule II using PLATON (Spek, A. L. (2009). Acta Cryst. D65, 148-155)/ORTEP (Johnson, C. K. (1976). ORTEPII. Report ORNL-5138. Oak Ridge National Laboratory, Tennessee, USA) at 30% probability level are shown in FIG. 7 and FIG. 8, respectively.

### Example 10

Fluctuation Ames Test

Each 20 μL of freeze-stored Salmonella typhimurium (TA98 and TA100 strain) was inoculated in 10 mL of liquid nutrient medium (2.5% Oxoid nutrient broth No. 2), and the cultures were incubated at 37° C. under shaking for 10 hours. 7.70 mL of TA98 culture was centrifuged (2000×g, 10 minutes) to remove medium, and the bacteria was suspended in 7.70 mL of Micro F buffer (K₂HPO₄: 3.5 g/L, KH₂PO₄: 1 g/L, (NH₄)₂ SO₄: 1 g/L, trisodium citrate dihydrate: 0.25 g/L, MgSO₄ 7H₂O: 0.1 g/L), and the suspension was added to 120 mL of Exposure medium (Micro F buffer containing Biotin: 8 μg/mL, histidine: 0.2 μg/mL, glucose: 8 mg/mL). 3.42 mL of TA100 culture was added to 130 mL of Exposure medium to prepare the test bacterial solution. 588 μL of the test bacterial solution (or mixed solution of 498 μL of the test bacterial solution and 90 μL of the S9 mix in the case with metabolic activation system) are mixed with each 12 μL of the following solution: DMSO solution of the test compound (several stage dilution from maximum dose 50 mg/mL at 2 to 3-fold ratio); DMSO as negative control; 50 μg/mL of 4-nitroquinoline-1-oxide DMSO solution as positive control for TA98 without metabolic activation system; 0.25 μg/mL of 2-(2-furyl)-3-(5-nitro-2-furyl)acrylamide DMSO solution as positive control for TA100 without metabolic activation system; 40 μg/mL of 2-aminoanthracene DMSO solution as positive control for TA98 with metabolic activation system; or 20 μg/mL of 2-aminoanthracene DMSO solution as positive control for TA100 with metabolic activation system. A mixed solution was incubated at 37° C. under shaking for 90 minutes. 460 μL of the bacterial solution exposed to the test compound was mixed with 2300 μL of Indicator medium (Micro F buffer containing biotin: 8 μg/mL, histidine: 0.2 μg/mL, glucose: 8 mg/mL, Bromo Cresol Purple: 37.5 μg/mL), each 50 μL was dispensed into 48 wells/dose in the microwell plates, and was subjected to stationary cultivation at 37° C. for 3 days. A well containing the bacteria, which has obtained the ability of proliferation by mutation in the gene coding amino acid (histidine) synthase, turns the color from purple to yellow due to pH change. The number of the yellow wells among the 48 total wells per dose was counted, and evaluate the mutagenicity by comparing with the negative control group. The results are shown in Table 12. (−) means that mutagenicity was negative.

TABLE 12

| | Structure | Mutagenicity |
|---|---|---|
| Compound 4 | 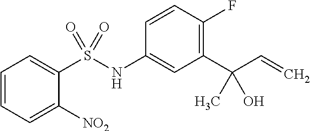 [SMILES: C=CC(C)(O)c1cc(NS(=O)(=O)c2ccccc2[N+](=O)[O-])ccc1F] | (−) |
| HBr salt of Compound 7 | 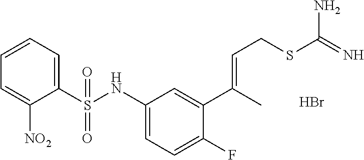 [SMILES: C/C(=C\CSC(=N)N)c1cc(NS(=O)(=O)c2ccccc2[N+](=O)[O-])ccc1F ; Br] | (−) |
| Anhydrate of Compound 9 | 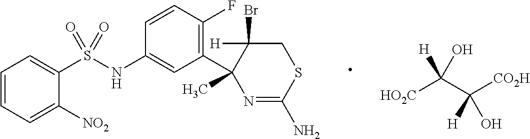 [SMILES: C[C@]1(c2cc(NS(=O)(=O)c3ccccc3[N+](=O)[O-])ccc2F)N=C(N)SC[C@@H]1Br ; O=C(O)[C@H](O)[C@@H](O)C(=O)O] | (−) |

### Example 11

Ames Test

Ames test is performed by using Salmonellas (Salmonella typhimurium) TA 98, TA100, TA1535 and TA1537 and Escherichia coli WP2uvrA as test strains to evaluate gene mutagenicity of the test compound. 0.1 mL of the test compound (DMSO solution) is mixed with 0.5 mL of S9 mix in the presence of metabolic activation or 0.5 mL of phosphate buffer in the absence of metabolic activation, and 0.1 mL of test strain suspension. The mixture is preincubated at 37° C. in the water bath for 20 minutes under shaking. After the preincubation, the mixture with 2 mL of layer soft agar, which contains histidine and biotin, or tryptophan, is overlaid on minimal glucose agar plates. Concurrently, negative control substance (DMSO) and positive control substance (2-(2-furyl)-3-(5-nitro-2-furyl)acrylamide, sodium azide, 9-aminoacridine, or 2-aminoanthracene) are also prepared. After incubation at 37° C. for 48 hours, the number of appeared revertant colonies are counted and evaluated by comparing with negative control group. It is judged to be positive when the number of revertant colonies is concentration-dependently increased and twofold or greater increased over the number of colonies of negative control group.

### INDUSTRIAL APPLICABILITY

The preparation methods and the compounds of the present invention are useful for the preparation of the pharmaceutical compound represented by formula (VI).

1. A process for preparing a compound of formula (II) or a salt thereof, 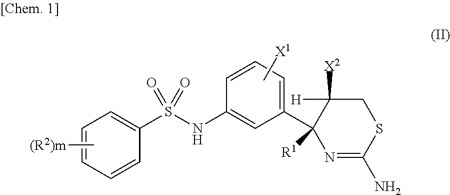
wherein
X¹ and X² are independently halogen;
R¹ is an optionally substituted alkyl;
R² is each independently NO₂, methyl, CF₃, halogen or methyloxy; and
m is an integer of 1 or 2,
which comprises subjecting a compound of formula (I) or a salt thereof, 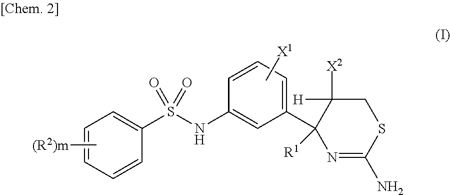
wherein each symbol is as defined above, to optical resolution using (L)-tartaric acid or (D)-malic acid.

2. The process of claim 1 wherein the optical resolution is carried out in a mixed solvent comprising water and one or more organic solvent selected from the group consisting of acetonitrile, methanol, 2-propanol, butanol, ethyl acetate, ethyl formate, acetone and methyl ethyl ketone.

3. The process of claim 2 wherein the mixed solvent comprises water, 2-propanol and ethyl acetate.

4. A process for preparing crystals of an acetate salt of a compound of formula (IV), 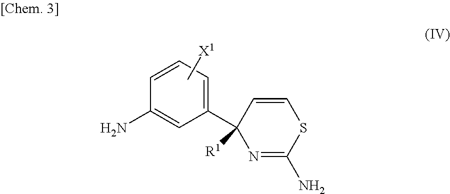
wherein
X¹ is halogen; and
R¹ is an optionally substituted alkyl,
which comprises the steps of:
treating a compound of formula (III) or a salt thereof, 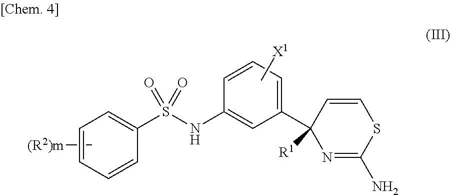
wherein each symbol is as defined above under acidic condition; and
subjecting the product to crystallization with acetic acid.

5. The process of claim 4 wherein the compound of formula (III) is that obtained by subjecting a compound of formula (II) or a salt thereof,
obtained by a process that comprises subjecting a compound of formula (I) or a salt thereof, 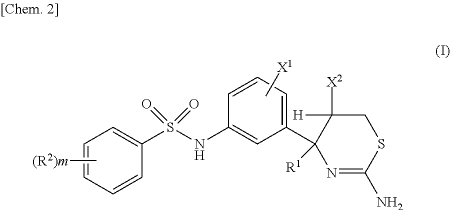
wherein X¹ and X² are independently halogen, R¹ is an optionally substituted alkyl, R² is each independently NO₂, methyl, CF₃, halogen or methyloxy, and m is an integer of 1 or 2, to optical resolution using (L)-tartaric acid or (D)-malic acid,
to dehydrohalogenation reaction.

6. A process for preparing a compound of formula (VI) or a salt thereof, 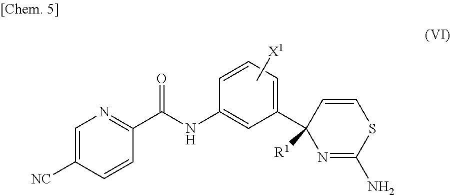
wherein
X¹ is halogen; and
R¹ is an optionally substituted alkyl,
which comprises reacting the acetate salt of a compound of formula (IV) obtained by the process of claim 4 or a free form thereof, 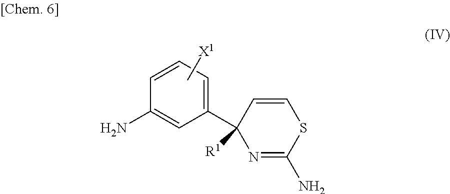
wherein each symbol is as defined above,
with a compound of formula (g): 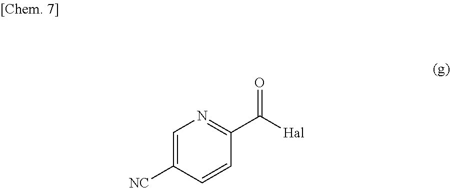
wherein Hal is halogen.

7. A process for preparing a compound of formula (VI) or a salt thereof, 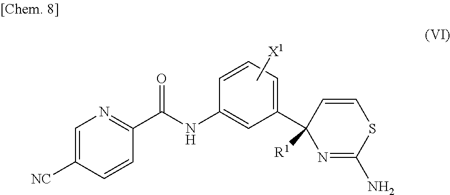
wherein
X¹ is halogen; and
R¹ is an optionally substituted alkyl,
which comprises the steps:
(1) subjecting a compound of formula (II) or a salt thereof to dehydrohalogenation reaction, 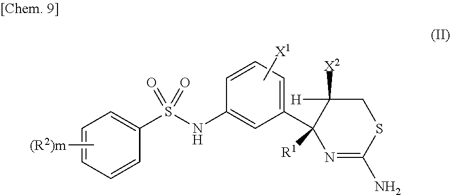
wherein
X¹ and R¹ are as defined above;
X² is halogen;
R² is each independently NO₂, methyl, CF₃, halogen or methyloxy; and
m is an integer of 1 or 2;
(2) deprotecting the product of formula (III) or a salt thereof, 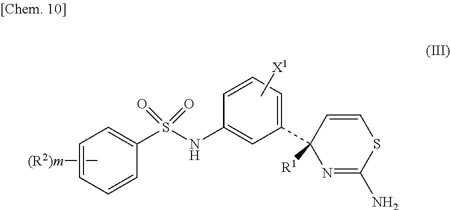
wherein each symbol is as defined above,
and
(3) reacting the product of formula (IV) or a salt thereof: 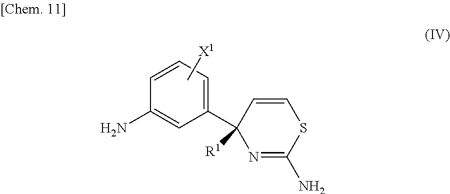
wherein each symbol is as defined above,
with a compound of formula (g): 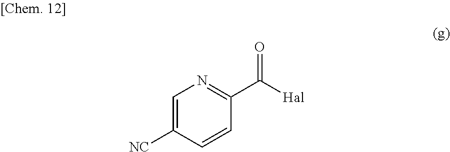
wherein Hal is as defined above.

8. The process of claim 7 wherein the compound of formula (II) or a salt thereof is obtained by a process comprising subjecting a compound of formula (I) or a salt thereof, 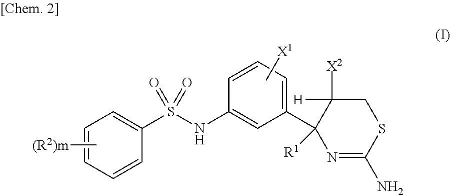
wherein X¹ and X² are independently halogen;
R¹ is an optionally substituted alkyl;
R² is each independently NO₂, methyl, CF₃, halogen or methyloxy; and
m is an integer of 1 or 2, each symbol is as defined above, to optical resolution using (L)-tartaric acid or (D)-malic acid.

9. A process for preparing a crystal of compound of formula (VI) or a salt thereof, 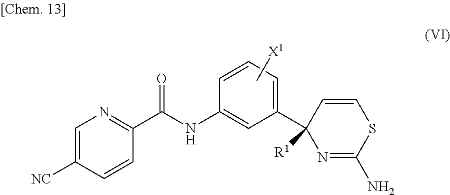
wherein
X¹ is halogen; and
R¹ is an optionally substituted alkyl,
which comprises the compound (VI) obtained by the process of claim 6 is neutralized with an organic base having 8 or more of pKa.

10. The process of claim 9 wherein the base is selected from the group consisting of alkylamine, dicyclohexylamine, ethanolamine, diethanolamine, triethanolamine, meglumine, ethylenediamine, and the mixture thereof.

11. The process of claim 9 wherein the base is triethylamine.

12. A compound of formula (VII) or a salt thereof, 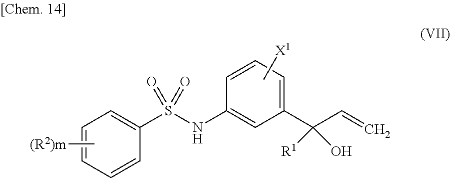
wherein
X¹ is halogen;
R¹ is an optionally substituted alkyl;
R² is each independently NO₂, methyl, CF₃, halogen or methyloxy; and
m is an integer of 1 or 2.

13. A compound of formula (VIII) or a salt thereof, 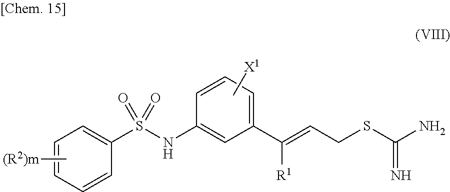
wherein
X¹ is halogen;
R¹ is an optionally substituted alkyl;
R² is each independently NO₂, methyl, CF₃, halogen or methyloxy; and
m is an integer of 1 or 2.

14. A compound of formula (II) or a salt thereof, 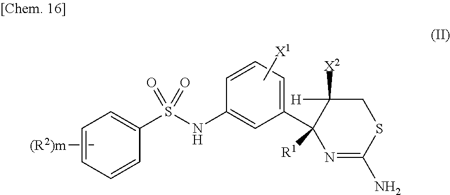
wherein
X¹ and X² are independently halogen;
R¹ is an optionally substituted alkyl;
R² is each independently NO₂, methyl, CF₃, halogen or methyloxy; and
m is an integer of 1 or 2.

15. The salt of claim 14 which is tartrate salt or malate salt.

16. A salt of a compound of formula (IV): 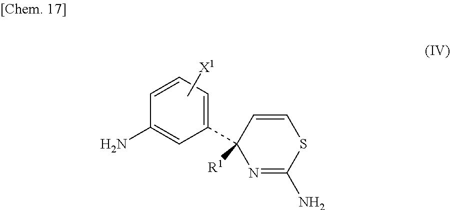
X¹ is halogen; and
R¹ is an optionally substituted alkyl.

17. The salt of claim 16 which is acetate salt.
